(19) United States
(12) Patent Application Publication
(10) Pub. No.: US 2026/0191892 A1
(43) Pub. Date: Jul. 9, 2026

(54) PHARMACEUTICAL COMPOSITION FOR TREATING ULCERATIVE COLITIS AND PREPARATION THEREOF

(71) Applicant: Centre for Chinese Herbal Medicine Drug Development Limited, Hong Kong (CN)

(72) Inventors: Zhaoxiang BIAN, Hong Kong (CN); Lin ZHU, Hong Kong (CN); Ka Wing CHENG, Hong Kong (CN); Hor Yue TAN, Hong Kong (CN); Jingchun SHI, Hong Kong (CN); Chengyuan Lin, Hong Kong (CN); Chunhua HUANG, Hong Kong (CN)

(21) Appl. No.: 19/553,657

(22) Filed: Mar. 2, 2026

Related U.S. Application Data
(63) Continuation-in-part of application No. 18/167,842, filed on Feb. 11, 2023.
(63) Continuation-in-part of application No. 18/462,749, filed on Sep. 7, 2023.

(30) Foreign Application Priority Data
Aug. 11, 2021 (CN) 202110927451.X
Jul. 26, 2023 (CN) 202310921375.0

(51) Int. Cl. A61K 31/7034; A61K 31/121; A61K 31/365; A61K 31/4375; A61K 31/7048; A61K 36/284; A61K 36/484; A61K 36/718; A61K 36/898; A61K 36/9066; A61P 1/04

(52) U.S. Cl. CPC A61K 31/7034; A61K 31/121; A61K 31/365; A61K 31/4375; A61K 31/7048; A61K 36/284; A61K 36/484; A61K 36/718; A61K 36/898; A61K 36/9066; A61P 1/04; A61K 2236/331; A61K 2236/333; A61K 2236/39

(57) ABSTRACT

The present invention provides a pharmaceutical composition including berberine, militarine, liquiritin, curcumin and atractylenolide III. The pharmaceutical composition is proven to be effective on ulcerative colitis modulating targets including MAOA, MAPK14, AHR, PTGS2, PLA2G1B, and ALOX5. The present invention further provides a method of preparing the pharmaceutical composition including optimization of extraction, determining the quality markers, and optimization of temperature. The present invention further provides a method of treating or alleviating ulcerative colitis.

### CROSS-REFERENCE TO RELATED APPLICATIONS

This application is a continuation-in-part (CIP) application of U.S. Non-Provisional patent application Ser. No. 18/167,842, filed on Feb. 11, 2023 and U.S. Non-Provisional patent application Ser. No. 18/462,749, filed on Sep. 7, 2023, and claims the benefit of priority of Chinese Patent Application Number 202110927451.X, filed on Aug. 11, 2021, and Chinese Patent Application Number 202310921375.0, filed on Jul. 26, 2023. The contents of which being hereby incorporated by reference in their entirety for all purposes.

### TECHNICAL FIELD

The present invention relates to a pharmaceutical composition for treating ulcerative colitis. The present invention also relates to a method of preparing a pharmaceutical composition for treating ulcerative colitis. The present invention also relates to a method of treating or alleviating ulcerative colitis by administering a pharmaceutical composition.

### BACKGROUND

Inflammatory bowel disease (IBD), including Crohn's disease and ulcerative colitis (UC), is characterized by intestinal inflammation, bloody stools, and diarrhea. UC is a chronic inflammatory disease that primarily involves the colonic mucosa, and its pathological process is closely associated with dysregulation of systemic metabolic networks and leads to aberrant activation of multiple molecular targets and signaling pathways. Current therapies for UC include 5-ASA, corticosteroids, immunomodulators, and biologic therapies that target inflammation and immune system dysregulation. 5-ASA inhibits the production of inflammatory chemicals and scavenges free radicals, while corticosteroids inhibit pro-inflammatory signaling pathways and immune cell activation. However, corticosteroids have serious adverse effects and should not be used long-term. Immunomodulators inhibit immune cell proliferation and regulate cytokine production. Biological agents, such as anti-TNF drugs, bind pro-inflammatory cytokines and reduce their effects.

A modified version of the Chinese medicinal formulation Si Jun Zi Tang comprises nine herbs: Codonopsis Radix, Poria, Atractylodis Macrocephalae Rhizoma, Glycyrrhizae Radix et Rhizoma, Paederiae Scandentis Herba et Radix, Bletillae Rhizoma, Curcumae Longae Rhizoma, Coptidis Rhizoma, and Corni Fructus. This formulation combines the tonifying and dampness-clearing herbs to address UC in patients with spleen deficiency and dampness-accumulation syndrome. The anti-colitis effect of this formulation has been confirmed in animal and human clinical trials without serious adverse effects. However, the active compounds, regulating targets, and pathways by which it alleviates UC remain unclear. Besides, hundreds of substances naturally exist in herbs of Chinese medicine, which makes the determination of the active ingredients and quality control difficult.

A need therefore exists for an improved composition including active ingredients that are confirmed to be effective for therapies of UC and the preparation method.

### SUMMARY OF INVENTION

Accordingly, a first aspect of the present invention provides a pharmaceutical composition comprising 40-70 wt. % berberine, 10-40 wt. % militarine, 10-25 wt. % liquiritin, 5-10 wt. % curcumin, and 0.01-1 wt. % atractylenolide III being characterized by molecular docking with an ulcerative colitis (UC)-modulating target selected from the group consisting of MAOA, MAPK14, AHR, PTGS2, PLA2G1B, ALOX5, and a combination thereof.

In certain embodiments, berberine, militarine, liquiritin, curcumin and atractylenolide III are respectively isolated from Coptidis Rhizoma, Bletillae Rhizoma, honey-processed Glycyrrhizae Radix et Rhizoma, Curcumae Longae Rhizoma, and bran-processed Atractylodis Macrocephalae Rhizoma.

In certain embodiments, berberine is at least 4.14 mg/g in the pharmaceutical composition.

In certain embodiments, militarine is at least 1.48 mg/g in the pharmaceutical composition.

In certain embodiments, berberine is 4.14-10.41 mg/g in the pharmaceutical composition.

In certain embodiments, militarine is 1.48-5.71 mg/g in the pharmaceutical composition.

In certain embodiments, berberine targets AHR, curcumin targets MAOA and MAPK14, atractylenoide III targets PTGS2, militarine targets ALOX5 and liquiritin targets PLA2G1B.

In certain embodiments, the pharmaceutical composition comprises 43.70-68.35 wt. % berberine, 11.75-37.45 wt. % militarine, 10.52-23.50 wt. % liquiritin, 5.26-9.08 wt. % curcumin, and 0.01-0.58 wt. % atractylenolide III.

In certain embodiments, berberine, militarine, liquiritin, curcumin and atractylenolide III are in weight ratio of 50:20:10:5:0.1.

In certain embodiments, the therapeutically effective amount of the pharmaceutical composition is 5.8-23.4 g/kg.

A second aspect of the present invention provides a method of treating or alleviating ulcerative colitis (UC) in a subject in need thereof, comprises administering a therapeutically effective amount of a composition consisting of berberine, militarine, liquiritin, curcumin and atractylenolide III being characterized by molecular docking with the UC-modulating targets consisting of MAOA, MAPK14, AHR, PTGS2, PLA2G1B, and ALOX5 to the subject in need thereof.

In certain embodiments, the composition consisting of 40-70 wt. % berberine, 10-40 wt. % militarine, 10-25 wt. % liquiritin, 5-10 wt. % curcumin, and 0.01-1 wt. % atractylenolide III.

In certain embodiments, the composition consisting of 43.70-68.35 wt. % berberine, 11.75-37.45 wt. % militarine, 10.52-23.50 wt. % liquiritin, 5.26-9.08 wt. % curcumin and 0.01-0.58 wt. % atractylenolide III.

In certain embodiments, berberine, militarine, glycyrrhizin, curcumin and atractylenolide III are in weight ratio of 50:20:10:5:0.1.

In certain embodiments, the therapeutically effective amount is 5.8-23.4 g/kg in a single dose or multiple doses daily.

In certain embodiments, the composition is administered via intragastrical route.

In certain embodiments, the subject is a human, a nonhuman primate, a farm animal or a domesticated mammal, or a laboratory animal.

A third aspect of the present invention provides a method for preparing the pharmaceutical composition comprising berberine, militarine, liquiritin, curcumin and atractylenolide III being isolated and characterized by molecular docking with an ulcerative colitis (UC)-modulating target selected from a group consisting of MAOA, MAPK14, AHR, PTGS2, PLA2G1B, ALOX5, and a combination thereof for treating or alleviating ulcerative colitis (UC), where the method comprises:
(a) characterizing which ingredients from a plurality of ingredients as active ingredients of the pharmaceutical composition by molecular docketing with the UC-modulating targets consisting of MAOA, MAPK14, AHR, PTGS2, PLA2G1B, and ALOX5;
(b) selecting berberine, militarine, liquiritin, curcumin and atractylenolide III based on the molecular docketing results obtained in (a); and
(c) providing and mixing 40-70 wt. % berberine, 10-40 wt. % militarine, 10-25 wt. % liquiritin, 5-10 wt. % curcumin, and 0.01-1 wt. % atractylenolide III. In certain embodiments, militarine, liquiritin, and atractylenolide III are isolated from Bletillae Rhizoma, honey-processed Glycyrrhizae Radix et Rhizoma and bran-processed Atractylodis Macrocephalae Rhizoma by water extraction respectively; and berberine and curcumin are isolated from Coptidis Rhizoma and Curcumae Longae Rhizoma by alcohol extraction respectively.

In certain embodiments, the water extraction is extracting 3 times with 8-10 times amount of water at a duration of 1 hour, and alcohol extraction is extracting 3 times with 8-10 times amount of 70% ethanol at a duration of 1-1.5 hours.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects of the present invention are disclosed as illustrated by the embodiments hereinafter.

### BRIEF DESCRIPTION OF DRAWINGS

The appended drawings, where like reference numerals refer to identical or functionally similar elements, include figures of certain embodiments to further illustrate and clarify the above and other aspects, advantages and features of the present invention. It will be appreciated that these drawings depict embodiments of the invention and are not intended to limit its scope. The invention will be described and explained with additional specificity and detail through the use of the accompanying drawings in which:

FIG. 1 schematically depicts the establishment of UC mouse model and in vivo evaluation on drugs according to an exemplary embodiment of the present invention;

FIG. 2A is a graph showing the body weight change of mice in control group (CTR), DSS group (DSS), positive control group (SSZ), CMF-L group, CMF-M group, and CMF-H group. Data are presented as the mean±S.D. (n=6-8). *, #: p<0.05, and **, ##: p<0.01; #: comparison between CTR group and DSS group; * comparisons between CMF/SSZ and DSS group.

FIG. 2B is a graph showing the change of Disease activity index (DAI) measured from mice in control group (CTR), DSS group (DSS), positive control group (SSZ), CMF-L group, CMF-M group, and CMF-H group. Data are presented as the mean±S.D. (n=6-8). *, #: p<0.05, and **, ##: p<0.01; comparison between CTR group and DSS group; *comparisons between CMF/SSZ and DSS group.

FIG. 2C is a graph showing the colon length of mice in control group (CTR), DSS group (DSS), positive control group (SSZ), CMF-L group, CMF-M group, and CMF-H group. Data are presented as the mean±S.D. (n=6-8). *: p<0.05, and **, ##: p<0.01; #: comparison between CTR group and DSS group; * comparisons between CMF/SSZ and DSS group.

FIG. 2D shows the images of mouse colon in (a) control group; (b) DSS group; (c) positive control group; (d) CMF-L group; (e) CMF-M group; and (f) CMF-H group.

FIG. 2E shows representative images of H&E staining (magnification, 4×) in (a) control group; (b) DSS group; (c) positive control group; (d) CMF-L group; (e) CMF-M group; and (f) CMF-H group. Red and blue arrows indicate epithelial destruction and inflammatory infiltration, respectively.

FIG. 3A is a graph showing colonic MPO levels in mice of control group (CTR), DSS group (DSS), positive control group (SSZ), CMF-L group, CMF-M group, and CMF-H group. Data are presented as the mean±S.D. (n=6-8). #p<0.05, ##p<0.01 vs. control group; *p<0.05, **p<0.01 vs. DSS group.

FIG. 3B is a graph showing the levels of pro-inflammatory cytokines, colonic TNF-α, in mice of control group (CTR), DSS group (DSS), positive control group (SSZ), CMF-L group, CMF-M group, and CMF-H group. Data are presented as the mean±S.D. (n=6-8). #p<0.05, ##p<0.01 vs. control group; *p<0.05, **p<0.01 vs. DSS group.

FIG. 3C is a graph showing the levels of pro-inflammatory cytokines, colonic IL-1β, in mice of control group (CTR), DSS group (DSS), positive control group (SSZ), CMF-L group, CMF-M group, and CMF-H group. Data are presented as the mean±S.D. (n=6-8). #p<0.05, ##p<0.01 vs. control group; *p<0.05, **p<0.01 vs. DSS group.

FIG. 3D is a graph showing the levels of pro-inflammatory cytokines, colonic IL-6, in mice of control group (CTR), DSS group (DSS), positive control group (SSZ), CMF-L group, CMF-M group, and CMF-H group. Data are presented as the mean±S.D. (n=6-8). #p<0.05, ##p<0.01 vs. control group; *p<0.05, **p<0.01 vs. DSS group.

FIG. 4A is a graph showing metabolic pathways identified in feces samples.

FIG. 4B is a graph showing metabolic pathways identified in serum samples.

FIG. 5 shows the concentrations of tryptophan, serotonin, tryptamine, indole acetic acid, linoleic acid, arachidonic acid, kynurenine, kynurenic acid, indole propionic acid, indole carboxaldehyde, phosphatidycholine, and lysophosphatidylcholine in fecal sample of control group (CTR), DSS group (DSS), positive control group (SSZ), CMF-L group, CMF-M group, and CMF-H group. Data are presented as the mean±S.D. (n=6-8). #p<0.05, p<0.01 vs. control group; *p<0.05, **p<0.01 vs. DSS group.

FIG. 6 shows the concentrations of tryptophan, serotonin, indole acetic acid, kynurenine, linoleic acid, arachidonic acid, kynurenic acid, indole propionic acid, indole carboxaldehyde, phosphatidycholine, and lysophosphatidylcholine in serum samples of control group (CTR), DSS group (DSS), positive control group (SSZ), CMF-L group, CMF-M group, and CMF-H group. Data are presented as the mean±S.D. (n=6-8). #p<0.05, p<0.01 vs. control group; *p<0.05, **p<0.01 vs. DSS group.

FIG. 7 is a diagram showing functional mechanism of CMF in UC remission.

FIG. 8A depicts molecular docking results between targets PTGS2, AHR, PLA2G6, and PLA2G1B and fifteen compounds (kJ/mol).

FIG. 8B depicts the molecular docking results of targets MAOA, MAPK14 and ALOX5.

FIG. 9A is Immunoblot results of colonic tissues in control group (CTR), DSS group (DSS), positive control group (SSZ), CMF-L group, CMF-M group, and CMF-H group.

FIG. 9B is a graph showing the relative protein expression of colonic tissues of control group (CTR), DSS group (DSS), positive control group (SSZ), CMF-L group, CMF-M group, and CMF-H group. Data are expressed as the mean and relative quantification of colonic tissues showed the inhibitory effects of CDD-2103 on COX-2 in DSS-treated mice. Data are expressed as the mean±S.D. (n=2). #p<0.05, ##p<0.01 vs. control group; *p<0.05, **p<0.01 vs. DSS group.

FIG. 9C is a graph showing the IC₅₀ values of CMF on COX-2 activities. Data are expressed as the mean±S.D. (n=3).

FIG. 9D is a graph showing the IC₅₀ values of berberine on COX-2 activities. Data are expressed as the mean±S.D. (n=3).

FIG. 9E is a graph showing the IC₅₀ values of curcumin on COX-2 activities. Data are expressed as the mean±S.D. (n=3).

FIG. 10A is a graph showing the levels of pro-inflammatory cytokines, TNF-α, in RAW264.7 macrophages of control group (CTR), LPS group (LPS), and C0-C8 groups. ####p<0.0001 vs. CTR; * p<0.05, ** p<0.01, *** p<0.001, and **** p<0.0001 vs. LPS; ††p<0.01, and †††p<0.001 vs. C0.

FIG. 10B is a graph showing the inhibitory rate on TNF-α in RAW264.7 macrophages of control group (CTR), LPS group (LPS), and C0-C8 groups.

FIG. 10C is a graph showing the levels of pro-inflammatory cytokines, IL-6, in RAW264.7 macrophages of control group (CTR), LPS group (LPS), and C0-C8 groups. ####p<0.0001 vs. CTR; * p<0.05, ** p<0.01, *** p<0.001, and **** p<0.0001 vs. LPS or C0.

FIG. 10D is a graph showing the inhibitory rate on IL-6 in RAW264.7 macrophages of control group (CTR), LPS group (LPS), and C0-C8 groups.

Skilled artisans will appreciate that elements in the figures are illustrated for simplicity and clarity and have not necessarily been depicted to scale.

### DETAILED DESCRIPTION OF THE INVENTION

It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

### Definitions

Throughout the present specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Furthermore, throughout the present specification and claims, unless the context requires otherwise, the word “include” or variations such as “includes” or “including”, will be understood to imply the inclusion of a stated integer or group of integers but not the exclusion of any other integer or group of integers.

The term “composition” is intended to encompass a product comprising the specified ingredients in the specified amounts, as well as any product that results, directly or indirectly, from combinations of the specified ingredients in the specified amounts.

As used herein, the term “isolated” in connection with a compound described herein means the compound is not in a botanical product and the compound is separated from some or all of the components that typically accompany it in a botanical product.

As used herein, the term “substantially pure” in connection with a sample of a compound described herein means the sample contains at least 60% by weight of the compound. In certain embodiments, the sample contains at least 70% by weight of the compound; at least 75% by weight of the compound; at least 80% by weight of the compound; at least 85% by weight of the compound; at least 90% by weight of the compound; at least 95% by weight of the compound; or at least 98% by weight of the compound.

As used herein, the terms “treat”, “treating”, “treatment”, and the like refer to reducing or ameliorating a disorder/disease and/or symptoms associated therewith. It will be appreciated, although not precluded, treating a disorder or condition does not require that the disorder, condition, or symptoms associated therewith be completely eliminated. In certain embodiments, treatment includes prevention of a disorder or condition, and/or symptoms associated therewith. The term “prevention” or “prevent” as used herein refers to any action that inhibits or at least delays the development of a disorder, condition, or symptoms associated therewith. Prevention can include primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

As used herein, the term “subject” refers to any animal (e.g., a mammal), including, but not limited to, humans, non-human primates, canines, felines, and rodents.

The term “therapeutically effective amount” as used herein, means that amount of active compound or pharmaceutical agent that elicits a biological or medicinal response in a cell culture, tissue system, animal, or human that is being sought by a researcher, veterinarian, clinician, or physician, which includes alleviation of the symptoms of the disease, condition, or disorder being treated.

The term “raw herbs” as used herein, (also referred to as crude herb(s), crude drug(s), herbal material, or decoction pieces/yinpian in TCM) means a plant- or fungus-derived material used in Chinese medicine, comprising the whole, cut, sliced, comminuted, or powdered aerial or subterranean parts (e.g., root, rhizome, stem, bark, leaf, flower, fruit, seed, or mycelium), fresh or dried, and not subjected to solvent extraction, fractionation, enrichment, or isolation of specific constituents. The term includes materials that have undergone non-extractive traditional preliminary processing (pao zhi) such as washing, sorting, trimming, sun- or shade-drying, steaming, baking, roasting or stir-frying (with or without adjuvants such as honey, wine, vinegar, or salt), carbonizing, and milling to a powder, provided that the material remains an intact crude matrix retaining its native spectrum of constituents. Unless expressly stated otherwise, “raw herb(s)” exclude purified or semi-purified compounds (e.g., isolated alkaloids or flavonoids), standardized extracts, tinctures, volatile oils/essential oils, fermented products with added microbial cultures, and finished dosage forms other than simple milled powders of the crude material. Authentication (e.g., botanical identity, part used, and quality attributes) may follow recognized pharmacopeial standards (e.g., Chinese Pharmacopoeia) and good sourcing practices.

Other definitions for selected terms used herein may be found within the detailed description of the present disclosure and apply throughout. Unless otherwise defined, all other technical terms used herein have the same meaning as commonly understood to one of ordinary skill in the art to which the invention belongs.

The present disclosure provides a pharmaceutical composition comprising berberine, militarine, glycyrrhizin, curcumin and atractylenolide III being isolated.

The pharmaceutical composition comprising berberine, militarine, liquiritin, curcumin and atractylenolide III being isolated can be administered to a subject either alone or in combination with pharmaceutically acceptable carriers or diluents in a pharmaceutical composition according to standard pharmaceutical practice. The pharmaceutical composition comprising berberine, militarine, liquiritin, curcumin and atractylenolide III being isolated can be administered orally or parenterally. Parenteral administration includes intravenous, intramuscular, intraperitoneal, subcutaneous and topical, the preferred method being intravenous administration.

Accordingly, the present disclosure provides pharmaceutically acceptable compositions, which comprise a therapeutically effective amount of the pharmaceutical composition comprising berberine, militarine, liquiritin, curcumin and atractylenolide III being isolated, formulated together with one or more pharmaceutically acceptable carriers (additives) and/or diluents. The pharmaceutical compositions of the present disclosure may be specially formulated for administration in solid or liquid form, including those adapted for the following: (1) parenteral administration, for example, by subcutaneous, intramuscular, intravenous or epidural injection as, for example, a sterile solution or suspension, or sustained-release formulation; and (2) oral administration, for example, drenches (aqueous or non-aqueous solutions or suspensions), tablets, e.g., those targeted for buccal, sublingual, and systemic absorption, boluses, powders, granules, pastes for application to the tongue.

Wetting agents, emulsifiers and lubricants, such as sodium lauryl sulfate and magnesium stearate, as well as coloring agents, release agents, coating agents, sweetening, flavoring and perfuming agents, preservatives, solubilizing agents, buffers and antioxidants can also be present in the compositions.

These compositions may also contain adjuvants, such as preservatives, wetting agents, emulsifying agents and dispersing agents. Prevention of the action of microorganisms upon the compounds of the present disclosure may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Disclosed herein is a metabolic network of UC and a composition including 5 active ingredients, berberine, militarine, liquiritin, curcumin and atractylenolide III, proven to be effective in UC metabolic network. Berberine, militarine, liquiritin, curcumin and atractylenolide III are confirmed to correspond to the UC key targets of MAPK14, PTGS2, ALOX5, MAOA, and AHR. Berberine, militarine, liquiritin, curcumin and atractylenolide III can reverse the abnormal metabolic changes caused by KA and IPA and increase phospholipid (PC) levels and reduces elevated lactate phosphate (LPC) and arachidonic acid (AA) levels in UC mouse model, which reveals the metabolism network between UC, metabolic pathways and active compounds. The composition including the 5 active ingredients exhibits anti-inflammatory effect and alleviating effect in UC mice model.

The weight ratio of berberine ranges from 40-70 wt. %, or 43.70-68.35 wt. % in the pharmaceutical composition. The weight ratio of militarine ranges from 10-40 wt. %, or 11.75-37.45 wt. % in pharmaceutical composition. The weight ratio of curcumin in pharmaceutical composition is 5-10 wt. %, or 5.26-9.08 wt. % in pharmaceutical composition. The weight ratio of liquiritin ranges from 10-25 wt. %, or 10.52-23.5 wt. %, or 10.52-13.33 wt. %, or 10.52-12.48 wt. % in the pharmaceutical composition. The weight ratio of atractylenolide III ranges from 0.01-1 wt. %, or 0.01-0.58 wt. %, or 0.01-0.12 wt. % in the pharmaceutical composition.

### EXAMPLES

### Example 1 —In Vivo Evaluation of CMF on UC Mouse Model

CMF includes Codonopsis Radix (Dang Shen, DS), Poria (Fu Ling, FL), bran-processed Atractylodis Macrocephalae Rhizoma (Fu Chao Bai Zhu, BZ), honey-processed Glycyrrhizae Radix et Rhizoma (Zhi Gan Cao, GC), Paederiae Scandentis Herba et Radix (Ji Shi Teng, JS), Bletillae Rhizoma (Bai Ji, BJ), Curcumae Longae Rhizoma (Jiang Huang, JH), Coptidis Rhizoma (Huang Lian, HL), and wine-processed Corni Fructus (Jiu Yu Rou, SY).

The powdered extract of CMF was provided by Beijing Increasepharm Corporation Limited (China). All herbs were authenticated by Pony Testing International Group (China) according to the Chinese Pharmacopoeia (version 2020) or Chinese Herbal Medicine Quality Standard (Hebei Province, version 2018). The ratio of DS:BZ:FL:SY:HL:JH:JS:BJ:GC was 4:2:4:1:1:1:2:2:1.

### 2.1 Experimental

The animal experiment was approved by the Committee on the Use of Human and Animal Subjects in Teaching and Research of Hong Kong Baptist University (REC/19-20/0301). Male C57BL6J mice aged 6-8 weeks were purchased from The Chinese University of Hong Kong (China). The mice were kept at a controlled temperature with a 12 h light/dark cycle with free access to water and food.

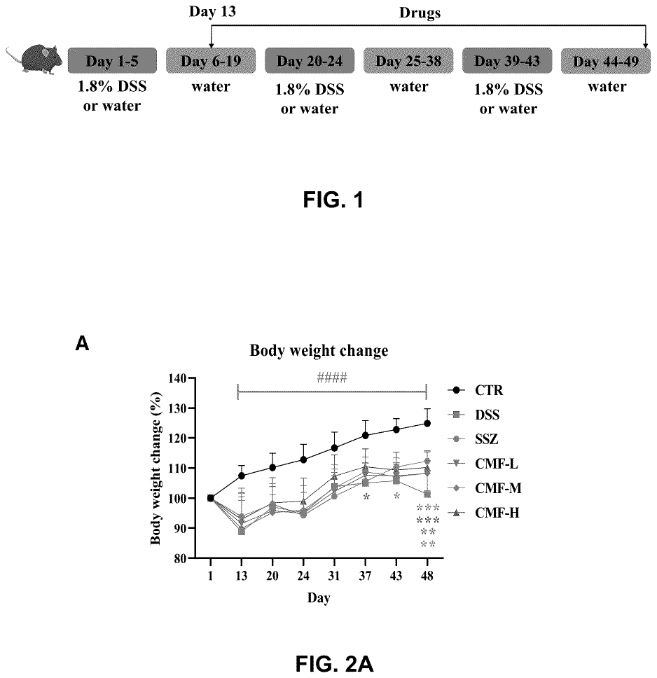

On Day 13, the mice were divided into six groups (n=8/group). One group is the control group without administration of any extra substances. The other five groups of mice were administered with water including 1.8% dextran sulfate sodium (DSS) for 3 cycles (49 days in total, FIG. 1) to induce chronic colitis, which showed slow weight gain, diarrhea and bloody stools. Three DSS-administered groups of mice were administered with CMF (CMF groups). A positive control group is established by sulfasalazine (SSZ). The detailed groups are as below:
CMF-L group: DSS-induced mice administered with low-dose CMF at 5.8 g/kg/day (based on raw herbs);
CMF-M group: DSS-induced mice administered with medium-dose CMF at 8.8 g/kg/day (based on raw herbs);
CMF-H group: DSS-induced mice administered with high-dose CMF at 11.7 g/kg/day (based on raw herbs);
Positive control group (SSZ): DSS-induced mice administered with SSZ at 500 mg/kg/day;
DSS group (DSS): DSS-induced mice without administering any medicine;
Control group (CTR): mice without administering DSS or any medicine.

The therapeutically effective amount of CMF for DSS-induced mice starts from 5.8 g/kg/day, which is converted from the therapeutically effective clinical dosage for humans (45 g/day based on raw herbs).

CMF and SSZ were orally administered to corresponding groups of mice, respectively, once per day starting from Day 13. The control and DSS groups were orally administered with an equal volume of water as in positive control and CMF groups. On Day 48, fecal samples of all groups were collected and kept at −80° C. until further use. On Day 49, mice were sacrificed, and serum and colon samples were collected.

### 2.2 Measurement of Body Weight, Disease Activity Index, Colon Length and Colonic Epithelial Destruction

The severity of colitis in mice was evaluated according to weight loss, the scoring of bloody stool, and stool consistency. Body weight change was calculated as the difference between the initial body weight on Day 1, and rated as follows: 0, <1% no loss; 1, 1-5% loss; 2, >5-10% loss; 3, >10-15% loss; 4, >15% loss. Hemoccult SENSA kits (USA) were used for bloody stool detection. Results were rated as follows: 0, no blood (brown); 1, some bleeding high in the gastrointestinal tract (slightly blue); 2, significant bleeding high in the gastrointestinal tract (blue); 3, slight bleeding (slightly red); 4, significant bleeding (red). Stool consistency was rated as follows: 0, normal; 1, soft but formed; 2, soft; 3, very soft and wet; 4, watery. Colon length was determined by measuring from the ileocecal junction to the anus.

After mice sacrifice, distal colons were rinsed and soaked in 4% paraformaldehyde at 4° C. overnight. Colonic tissues were sequentially dehydrated and finally embedded in paraffin blocks. Tissues were segmented into 4 m slices and stained (H&E) for the observation of colonic injuries.

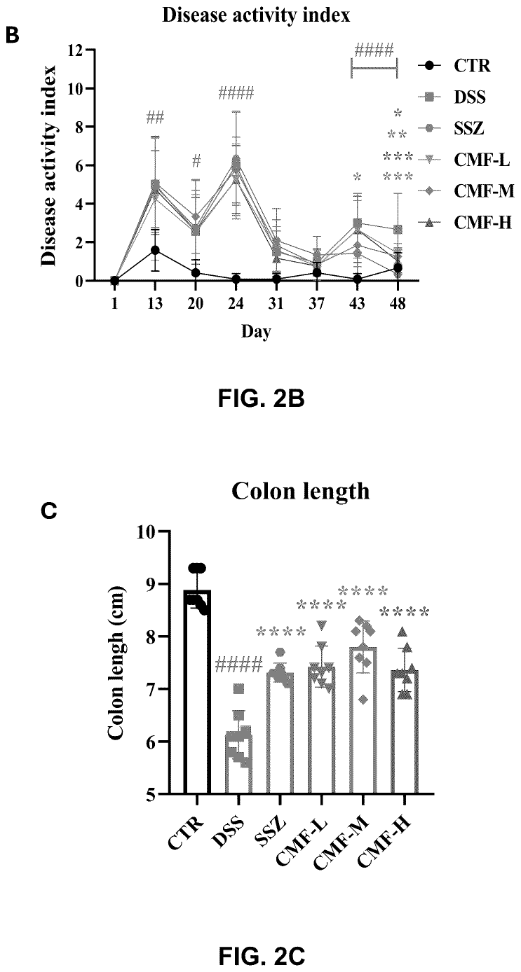

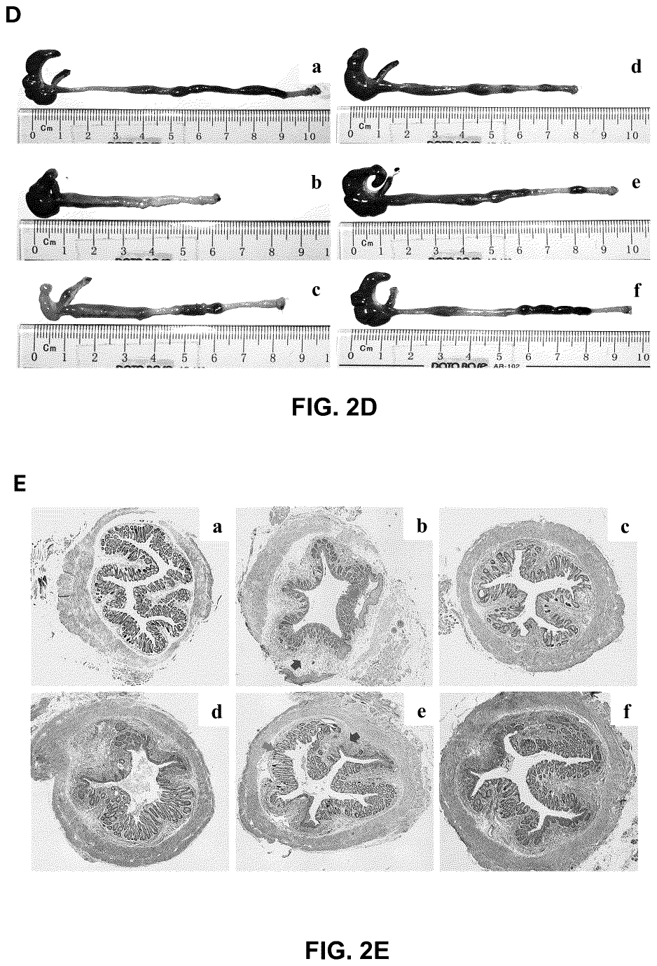

Body weight loss, disease activity index (DAI), and colonic shortening induced by DSS in mice were remarkably alleviated by CMF (FIGS. 2A-2D). CMF protected mice from severe epithelial destruction and inflammatory cell infiltration caused by colitis (FIG. 2E). These results showed that CMF groups of all dosages exhibit comparable or better efficacy than SSZ group.

### 2.3 Measurements on Cytokines and MPO Activity of CMF

The colonic tissues were homogenized in RIPA buffer including protease inhibitor cocktail (Solarbio, China). The supernatant was collected as total protein samples after centrifugation at 12,000 rpm at 4° C. Protein concentrations were examined using the BCA Kit (Thermo Fisher Scientific, USA). IL-1β, IL-6, TNF-α, and myeloperoxidase (MPO) levels in the homogenates were testified using ELISA kits according to the standard procedures recommended by Thermo Fisher Scientific company. The results were normalized to the total weight of colon tissues and quantified as pg/mg.

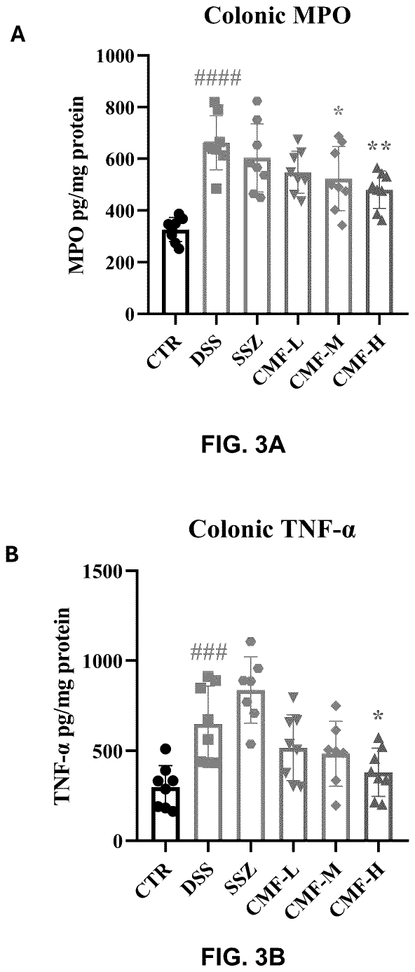

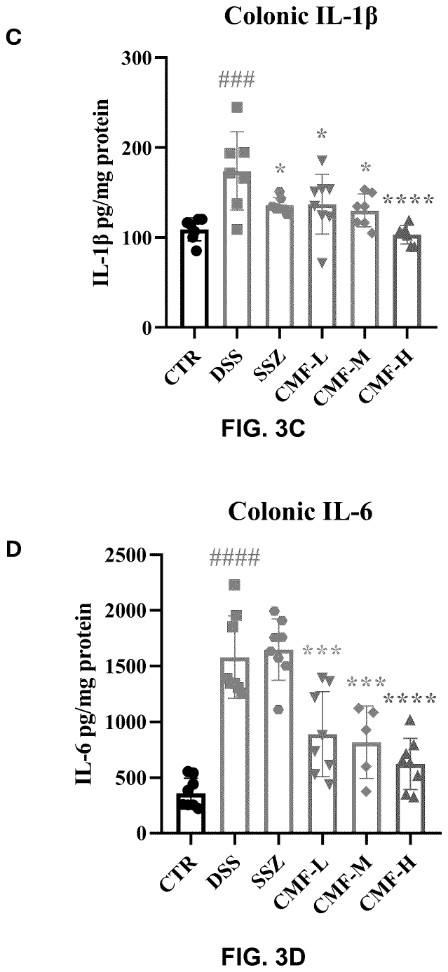

CMF treatment significantly reduced the elevated levels of MPO activity and pro-inflammatory cytokines (IL-1β, TNF-α, and IL-6), while SSZ specifically showed a therapeutic effect on IL-1β. CMF significantly ameliorated DSS-induced symptoms, which demonstrated its anti-inflammatory and anti-colitis effects (FIGS. 3A-3D).

### Example 2—Screening UC-Related Pathways and Biomarkers

### 2.1 Metabolism Analysis

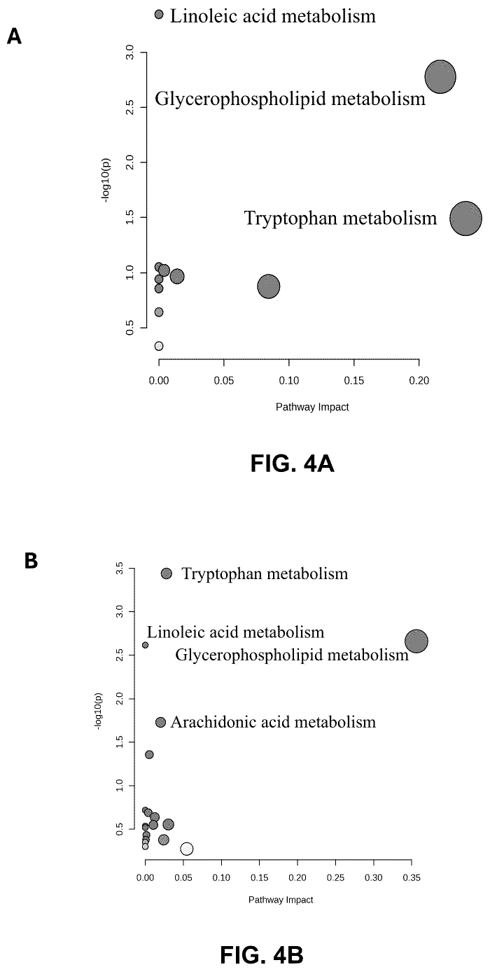

UC (DSS inducement) and CMF treatment caused significant metabolic changes in mice. To reveal the relationship between UC and metabolites, a total of 17,735 and 7702 features were determined in all fecal and serum samples, respectively. Among them, 37 metabolites in feces (Table 1) and 17 metabolites in serum (Table 2) were identified to have altered in the DSS group but reversed by administering CMF in both fecal and serum samples. The inventors further imported these differential metabolites to MetaboAnalyst 4.0. Combining FDR>0.05 and/or impact >0.1, as shown in FIGS. 4A and 4B, three pathways were affected significantly in the feces and serum samples, namely tryptophan, linoleic acid, and glycerophospholipid metabolisms. Key metabolites involved in these pathways included arachidonic acid, linoleic acid, L-tryptophan, and phosphatidylcholine.

TABLE 1
Detailed information on differential metabolites in fecal samples.

| RT (min) | Precursor MS | MS/MS | Compound name | HMDB ID | FC1 | FC2 | P (1) | P (2) |
|---|---|---|---|---|---|---|---|---|
| 0.9 | 195.0480 | 123.0087 | Pectic acid | HMDB0003363 | 2.31 | 0.43 | 0.001 | 0.003 |
| 1.4 | 134.0472 | 104.0500 | Adenine | HMDB0000034 | 2.84 | 0.39 | 0.024 | 0.037 |
| 1.8 | 157.0507 | 227.2017 | Isopropylmaleic acid | HMDB0012241 | 0.54 | 1.75 | 0.001 | 0.004 |
| 2.1 | 445.1876 | 152.9958 | Estrone glucuronide | HMDB0004483 | 3.96 | 0.58 | 0.001 | 0.045 |
| 2.8 | 136.0745 | 96.9696 | 2-Phenylacetamide | HMDB0010715 | 2.17 | 0.59 | 0.000 | 0.010 |
| 2.8 | 252.1087 | 205.1961 | Deoxyadenosine | HMDB0000101 | 1.67 | 0.38 | 0.045 | 0.006 |
| 2.9 | 185.0810 | 295.1698 | Vanylglycol | HMDB0001490 | 1.60 | 0.55 | 0.027 | 0.014 |
| 3.5 | 140.0353 | 96.9696 | 2-Aminomuconic acid semialdehyde | HMDB0001280 | 2.79 | 0.32 | 0.018 | 0.023 |
| 3.9 | 130.0661 | 71.0133 | 3-Methylindole | HMDB0000466 | 6.59 | 0.05 | 0.037 | 0.033 |
| 5.0 | 568.3362 | 327.2329 | LysoPC (22:5(4Z, 7Z, 10Z, 13Z, 16Z)/0:0) | HMDB0010402 | 0.59 | 1.67 | 0.047 | 0.007 |
| 5.5 | 466.2921 | 132.0454 | LysoPC (14:0/0:0) | HMDB0010379 | 1.31 | 0.60 | 0.049 | 0.001 |
| 5.6 | 170.0591 | 90.0343 | Phosphodimethylethanolamine | HMDB0060244 | 0.50 | 2.16 | 0.017 | 0.012 |
| 5.7 | 160.0405 | 174.0415 | Quinoline-4,8-diol | HMDB0060289 | 0.13 | 8.23 | 0.017 | 0.010 |
| 5.7 | 116.0508 | 277.2173 | Indole | HMDB0000738 | 0.19 | 9.00 | 0.013 | 0.000 |
| 6.1 | 162.0903 | 98.9847 | Tryptophol | HMDB0003447 | 0.25 | 2.16 | 0.021 | 0.044 |
| 6.7 | 437.2666 | 138.9796 | LysoPA (18:0/0:0) | HMDB0007854 | 1.90 | 0.63 | 0.002 | 0.034 |
| 6.7 | 426.0114 | 44.9976/133.0136 | Adenosine phosphosulfate | HMDB0001003 | 0.69 | 1.43 | 0.006 | 0.007 |
| 8.1 | 482.3262 | 91.0542/65.0386 | LysoPE (18:0/0:0) | HMDB0011130 | 2.36 | 0.27 | 0.026 | 0.011 |
| 8.3 | 965.5226 | 67.0183/210.0878 | PIP (22:5(4Z, 7Z, 10Z, 13Z, 16Z)/16:0) | HMDB0010029 | 2.31 | 0.25 | 0.040 | 0.016 |
| 9.4 | 411.2483 | 69.0340/167.0708 | LysoPA (16:0/0:0) | HMDB0007853 | 3.62 | 0.23 | 0.046 | 0.041 |
| 9.5 | 87.0432 | 43.0542/341.3050 | Diacetyl | HMDB0003407 | 0.64 | 2.26 | 0.021 | 0.002 |
| 10.2 | 279.2303 | 711.2905/659.3167 | Gamma-Linolenic acid | HMDB0003073 | 0.34 | 21.59 | 0.042 | 0.019 |
| 10.8 | 319.2276 | 43.0547/73.0289 | 12-KETE | HMDB0013633 | 0.22 | 5.20 | 0.013 | 0.002 |
| 11.1 | 758.6021 | 261.2576 | PE (22:0/P-16:0) | HMDB0009510 | 1.49 | 0.39 | 0.000 | 0.000 |
| 11.2 | 319.2276 | 163.1117 | 5,6-Epoxy-8,11,14-eicosatrienoic acid | HMDB0002190 | 3.88 | 0.49 | 0.001 | 0.019 |
| 11.7 | 756.5477 | 60.0807/712.4911 | PC (14:0/20:3(8Z, 11Z, 14Z)) | HMDB0007882 | 16.54 | 0.01 | 0.007 | 0.009 |
| 11.7 | 139.1110 | 72.0807/80.9736 | (3Z, 6Z)-3,6-Nonadienal | HMDB0031152 | 0.31 | 1.90 | 0.001 | 0.003 |
| 11.8 | 647.5075 | 144.0801 | SM(d18:1/12:0) | HMDB0012096 | 0.33 | 2.54 | 0.000 | 0.003 |
| 11.9 | 401.3395 | 45.0340/51.0234 | 7a-Hydroxy-cholestene-3-one | HMDB0001993 | 0.62 | 2.76 | 0.026 | 0.000 |
| 12.0 | 343.1892 | 189.1643 | 11-Dehydrocorticosterone | HMDB0004029 | 2.59 | 0.56 | 0.001 | 0.018 |
| 12.0 | 729.5899 | 175.1117 | SM(d18:0/18:1(11Z)) | HMDB0012088 | 2.28 | 0.27 | 0.009 | 0.006 |
| 12.1 | 717.5526 | 105.0704 | SM (00d18:0/16:1(9Z) (OH)) | HMDB0013463 | 0.11 | 2.69 | 0.034 | 0.010 |
| 12.5 | 291.2302 | 464.4462 | 4-Methylphenyl dodecanoate | HMDB0037711 | 0.49 | 1.92 | 0.004 | 0.045 |
| 12.7 | 393.3003 | 69.0704/311.2374 | Murocholic acid | HMDB0000811 | 2.24 | 0.31 | 0.049 | 0.028 |
| 12.8 | 349.2379 | 631.4445 | Tetrahydrocorticosterone | HMDB0000268 | 0.49 | 1.55 | 0.004 | 0.043 |
| 13.0 | 756.5477 | 135.1173 | PC (20:3(8Z, 11Z, 14Z)/14:0) | HMDB0008394 | 12.26 | 0.01 | 0.000 | 0.000 |
| 13.3 | 377.2344 | 69.0698/161.1324 | 14-Hydroxy-E4-neuroprostane | HMDB0012580 | 0.14 | 6.49 | 0.000 | 0.000 |

FC1: comparison between the DSS and CTR groups, FC2: comparison between the CMF-treated and DSS groups

TABLE 2
Detailed information on differential metabolites in serum samples.

| RT | Precursor | | | | | | | |
|---|---|---|---|---|---|---|---|---|
| (min) | MS | MS/MS | Compound name | HMDB ID | FC1 | FC2 | P (1) | P (2) |
| 1.6 | 177.1035 | 160.0746 | Serotonin | HMDB0000259 | 0.52 | 2.43 | 0.001 | 0.000 |
| 2.0 | 221.0923 | 155.0609 | 5-Hydroxy-L-tryptophan | HMDB0000472 | 0.32 | 3.10 | 0.004 | 0.001 |
| 3.7 | 477.2479 | 105.0704 | Retinoyl b-glucuronide | HMDB0003141 | 0.42 | 1.64 | 0.000 | 0.015 |
| 4.8 | 817.7204 | 619.4809 | SM(d18:0/24:0) | HMDB0012094 | 1.61 | 0.58 | 0.029 | 0.011 |
| 5.4 | 835.7694 | 852.8020 | TG (16:0/16:0/18:0) | HMDB0005357 | 1.40 | 0.71 | 0.014 | 0.015 |
| 6.3 | 714.5378 | 86.0964/574.3867 | PC (14:1(9Z)/P-18:1(9Z)) | HMDB0007931 | 3.33 | 0.65 | 0.031 | 0.031 |
| 11.1 | 754.5362 | 72.0807/696.4598 | PC (20:4(8Z, 11Z, 14Z, | HMDB0008459 | 0.44 | 8.43 | 0.003 | 0.000 |
| | | | 17Z)/14:0) | | | | | |
| 11.2 | 548.3700 | 75.0440/365.3050 | LysoPC (20:2(11Z, 14Z)/0:0) | HMDB0010392 | 0.45 | 2.26 | 0.001 | 0.008 |
| 11.5 | 297.2416 | 153.1273 | 9,10-Epoxyoctadecenoic acid | HMDB0004701 | 3.17 | 0.67 | 0.007 | 0.022 |
| 12.0 | 758.5691 | 617.5508 | PE (22:2(13Z, 16Z)/15:0) | HMDB0009549 | 1022 | 0.28 | 0.019 | 0.010 |
| 12.2 | 717.5548 | 659.4758 | SM (d18:0/16:1(9Z) (OH)) | HMDB0013463 | 2.19 | 0.53 | 0.049 | 0.045 |
| 12.5 | 317.2470 | 91.0542 | Alpha-dihydroprogesterone | HMDB0003069 | 1.85 | 0.33 | 0.017 | 0.000 |
| 12.7 | 736.4876 | 285.2429 | PE (22:6(4Z, 7Z, 10Z, 13Z, | HMDB0009679 | 0.23 | 2.18 | 0.000 | 0.000 |
| | | | 16Z, 19Z)/14:0) | | | | | |
| 12.9 | 303.2330 | 193.0512 | Arachidonic acid | HMDB0001043 | 1.75 | 0.33 | 0.011 | 0.000 |
| 12.7 | 550.3858 | 184.0733 | PC (18:1(9Z) e/2:0) | HMDB0011148 | 2.36 | 0.33 | 0.029 | 0.005 |
| 13.0 | 279.2330 | 128.0099 | Linoleic acid | HMDB0000673 | 0.25 | 2.18 | 0.001 | 0.000 |
| 15.4 | 951.7379 | 881.6654 | TG (20:4(5Z, 8Z, 11Z, 14Z)/ | HMDB0005478 | 0.21 | 50.16 | 0.031 | 0.000 |
| | | | 20:4(5Z, 8Z, 11Z, 14Z) | | | | | |

FC1: comparison between the DSS and CTR groups,
FC2: comparison between the CMF-treated and DSS groups

### 2.2 Quantification Analysis

To further investigate the alterations observed in tryptophan, linoleic acid, and glycerophospholipid metabolites, the concentrations of the potentially relevant metabolites in fecal and serum samples are measured.

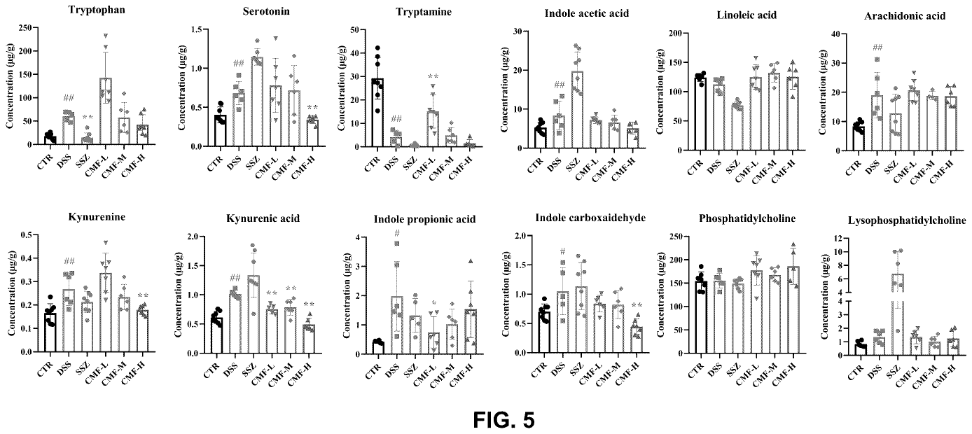

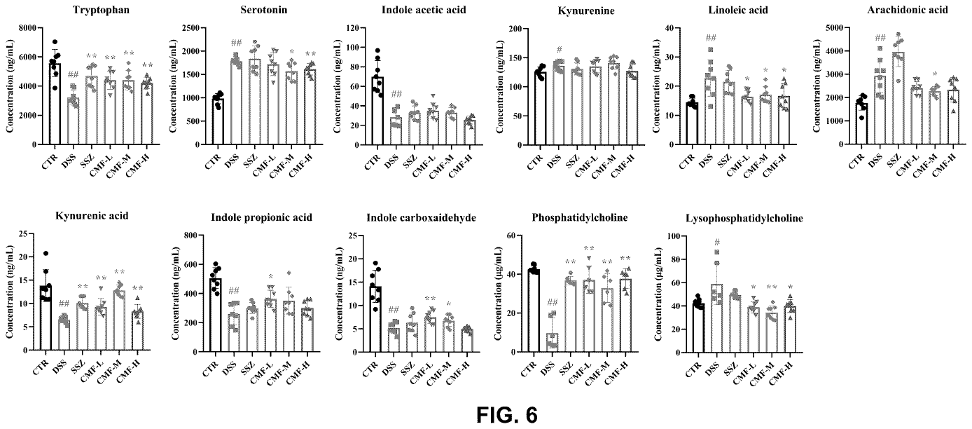

In fecal samples, all tryptophan metabolites were significantly upregulated in the DSS group compared to the control group, except for tryptamine (TM) as shown in FIG. 5. All these metabolic disorders were reversed to some extent after treatment with CMF, while SSZ only showed the therapeutic effect on tryptophan. Among linoleic acid and glycerophospholipid metabolites, only arachidonic acid exhibited a significant difference between the DSS and control groups. In serum samples, there were no significant differences in kynurenine levels among groups, and TM was undetectable (FIG. 6). However, apart from serotonin, all other tryptophan metabolites were significantly downregulated in the DSS group compared to the control group.

Treatment with CMF and SSZ restored these altered conditions. Furthermore, except for phosphatidylcholine (PC), all other glycerophospholipid and linoleic acid metabolites were significantly upregulated in the DSS group compared to control group. CMF treatment effectively reversed these metabolic disruptions, while SSZ primarily influenced PC levels. The effectiveness of CMF on glycerophospholipid and linoleic acid metabolites only appears in serum samples, suggesting that CMF influences host-derived metabolites within these metabolic pathways, while CMF affects both host and bacterial-derived tryptophan metabolites.

Tryptophan, glycerophospholipid, and linoleic acid metabolisms might be the critical metabolic pathways and that kynurenic acid (KA), phosphatidylcholine (PC), and arachidonic acid (AA) were the related vital metabolites and biomarkers. Based on the important role of these targets in UC and their interaction with CMF compounds, three core targets, AHR, PLA2, and PTGS2, were extracted.

### Example 3—Identification of Active Ingredients from CMF

### 3.1 Serum and Feces Component Analysis and Network Pharmacology

The serum and fecal samples of CMF groups of Example 2 were respectively extracted and analyzed by UPLC-Q-TOF/MS. The compounds of CMF identified in the serum and fecal samples are summarized in Table 3. Components were identified using accurate m z, fragmentation data, or reference standards. 23 compounds, including prototypes and metabolites, identified in the serum-feces pharmaco-chemistry study were subjected to network pharmacology study.

TABLE 3
compounds of CMF identified in the serum and fecal samples by UPLC-Q-TOF/MS

| | | | | Positive ion mode | | | |
|---|---|---|---|---|---|---|---|
| | | | | | | Mass | |
| | RT | | | | | accuracy | MS/MS |
| No | (min) | Formula | Compound | Adduct | m/z | (ppm) | (fragments) |
| 1 | 3.56 | C₁₇H₂₆O₁₁ | Morroniside* | [M + Na]⁺ | 429.1358 | −2.1 | 385/267 |
| 2 | 5.64 | C₁₇H₂₆O₁₀ | Loganin* | / | / | / | / |
| 3 | 6.00 | C₂₀H₂₄NO₄⁺ | Magnoflorine* | [M]⁺ | 342.1696 | −2.63 | 297/265/237 |
| 4 | 6.44 | C₈H₁₄O₅ | α-Isobutylmalic acid | / | / | / | / |
| 5 | 6.48 | C₆H₁₂O | Gamma-Hexenol | / | / | / | / |
| 6 | 8.91 | C₂₆H₂₈NO₁₀⁺ | Jatrorrhizine-3-O- | [M]⁺ | 514.1706 | −1.36 | 485/434/417 |
| | | | beta-D-glucuronide | | | | |
| 7 | 9.86 | C₂₁H₂₂O₉ | Glycyrrhizin* | / | / | / | / |
| 8 | 11.16 | C₁₉H₁₆NO₄⁺ | Berberrubine* | [M]⁺ | 322.1069 | −3.1 | 307/279 |
| 9 | 11.85 | C₆H₁₂ | Methylcyclopentane | / | / | / | / |
| 10 | 14.25 | C₁₉H₁₄NO₄⁺ | Coptisine* | [M]⁺ | 320.0914 | −2.81 | 305/292/277 |
| 11 | 14.31 | C₂₀H₂₀NO₄⁺ | Columbamine* | [M]⁺ | 338.1382 | −2.96 | 323/294 |
| 12 | 14.43 | C₂₀H₁₈NO₄⁺ | Epiberberine* | [M]⁺ | 336.1229 | −2.08 | 320/292 |
| 13 | 14.78 | C₂₀H₂₀NO₄⁺ | Jatrorrhizine* | [M]⁺ | 338.1384 | −2.37 | 323/294 |
| 14 | 18.05 | C₂₀H₁₈NO₄⁺ | Berberine* | [M]⁺ | 336.1229 | −2.08 | 321/292 |
| 15 | 18.05 | C₂₁H₂₂NO₄⁺ | Palmatine* | [M]⁺ | 352.1542 | −1.99 | 337/322/308 |
| 16 | 18.07 | C₃₄H₄₆O₁₇ | Militarine* | / | / | / | / |
| 17 | 19.06 | C₂₀H₁₆NO₇ | Cop+ 3hydroxylation+ | [M]⁺ | 382.092 | 1.83 | 364/345/318/297 |
| | | | methylation | | | | |
| 18 | 21.02 | C₄₂H₆₂O₁₆ | Glycyrrhizic acid* | [M + H]⁺ | 823.4091 | −2.43 | 648/472/454 |
| 19 | 22.30 | C₁₉H₁₆O₄ | Bisdemethoxycurcumin* | [M + H]⁺ | 309.1121 | 0.00 | 225/189/147 |
| 20 | 22.54 | C₁₅H₂₀O₃ | Atractylenolide III* | [M + H]⁺ | 249.1486 | −2.02 | 229/203/187 |
| 21 | 22.60 | C₂₀H₁₈O₅ | Demethoxycurcumin* | [M + H]⁺ | 339.1223 | −1.18 | 255/177/147 |
| 22 | 22.86 | C₂₁H₂₀O₆ | Curcumin* | [M + H]⁺ | 369.1335 | 0.54 | 285/177 |
| 23 | 25.02 | C₃₂H₄₈O₆ | Poricoic acid DM | / | / | / | / |

| | Negative ion mode | | | | | | | |
|---|---|---|---|---|---|---|---|---|
| | | | Mass | | | | | |
| | | | accuracy | MS/MS | In | In | | |
| No | Adduct | m/z | (ppm) | (fragments) | serum | feces | Type | Origin |
| 1 | [M + COOH]⁻ | 451.1462 | 1.11 | 405/243/141 | + | / | P | SY |
| 2 | [M + COOH]⁻ | 435.1491 | −3.91 | 227/101 | + | / | P | SY |
| 3 | / | / | / | / | + | + | P | HL |
| 4 | [M − H]⁻ | 189.0772 | 2.12 | 171/145/129 | + | + | P | BJ |
| 5 | [M + COOH]⁻ | 145.0872 | 1.38 | 134/119/103 | / | + | M | JS |
| 6 | / | / | / | / | + | / | M | HL |
| 7 | [M − H]⁻ | 417.1195 | 0.96 | 255/135 | + | / | P | GC |
| 8 | / | / | / | / | + | + | P | HL |
| 9 | [M + COOH]⁻ | 129.0927 | 4.65 | 116/101 | / | + | M | GC |
| 10 | / | / | / | / | + | + | P | HL |
| 11 | / | / | / | / | + | + | P | HL |
| 12 | / | / | / | / | + | + | P | HL |
| 13 | / | / | / | / | + | + | P | HL |
| 14 | / | / | / | / | + | + | P | HL |
| 15 | / | / | / | / | + | + | P | HL |
| 16 | [M + COOH]⁻ | 771.2700 | −2.72 | 726/457 | + | + | P | BJ |
| 17 | / | / | / | / | + | / | M | HL |
| 18 | / | / | / | / | + | + | P | GC |
| 19 | [M − H]⁻ | 307.0966 | −3.26 | 187/143/119 | / | + | P | JH |
| 20 | [M − H]⁻ | 247.1336 | −1.62 | 203/187 | / | + | P | BZ |
| 21 | [M − H]⁻ | 337.1077 | −1.19 | 217/173/119 | / | + | P | JH |
| 22 | [M − H]⁻ | 367.1186 | −0.27 | 217/173/149 | / | + | P | JH |
| 23 | [M − H]⁻ | 527.3373 | −0.95 | 485/467/423 | / | + | M | FL |

“*” represents the compounds determined by comparison with reference standards;
“P” represents prototype, and “M” represents metabolite;
SY: wine-processed Corni Fructus;
HL: Coptidis Rhizoma;
BJ: Bletillae Rhizoma;
JS: Paederiae Scandentis Herba et Radix;
GC: honey-processed Glycyrrhizae Radix et Rhizoma;
JH: Curcumae Longae Rhizoma;
BZ: bran-processed Atractylodis Macrocephalae Rhizoma;
FL: Poria

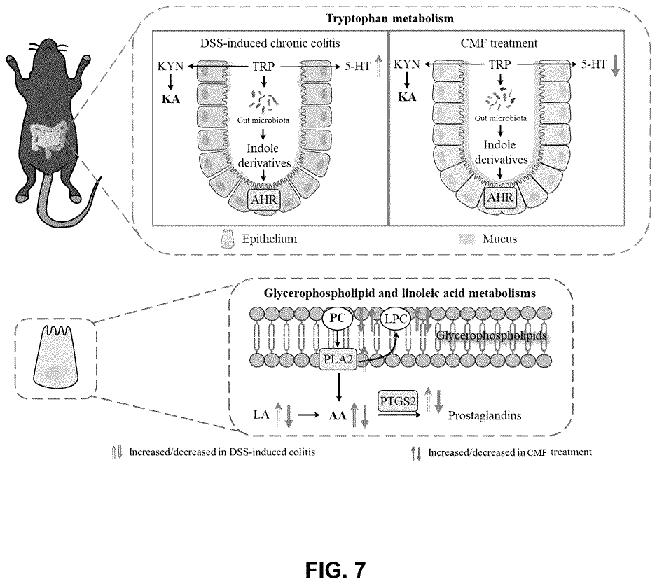

Based on the differential metabolites and the potential targets, the compound-reaction-enzyme-gene network showed that MAOA, AHR, MAPK14, PLA2G1B, ALOX5, and PTGS2 might be the key targets, and that, KA, phosphatidylcholine (PC), and arachidonic acid (AA) might be the key metabolites regulated by CMF (FIG. 7). Based on the herb-compound-target-pathway-metabolite-disease network, the inventors found that tryptophan, glycerophospholipid and linoleic acid metabolisms might be the key metabolic pathways, and that, curcumin, berberine, atractylenolide III, liquiritin, and militarine might be the key active ingredients (Table 4).

TABLE 4
Key active ingredients, targets, metabolites, and pathways
for CMF in UC remission as revealed by integrated
analysis of metabolomics and network pharmacology.

| Pathway | Metabolite | Target | Active compound |
|---|---|---|---|
| Tryptophan | KA | MAOA, | Curcumin, berberine |
| metabolism | | AHR | |
| Glycerophospholipid | PC | PLA2G1B, | Liquiritin, Atractylenoide |
| metabolism | | MAPK14 | III, berberine, curcumin |
| Linoleic acid | AA | PTGS2, | Atractylenoide |
| metabolism | | ALOX5 | III, militarine |

KA: kynurenic acid;
PC: phosphatidylcholine;
AA: arachidonic acid

### 3.2 molecular Docketing

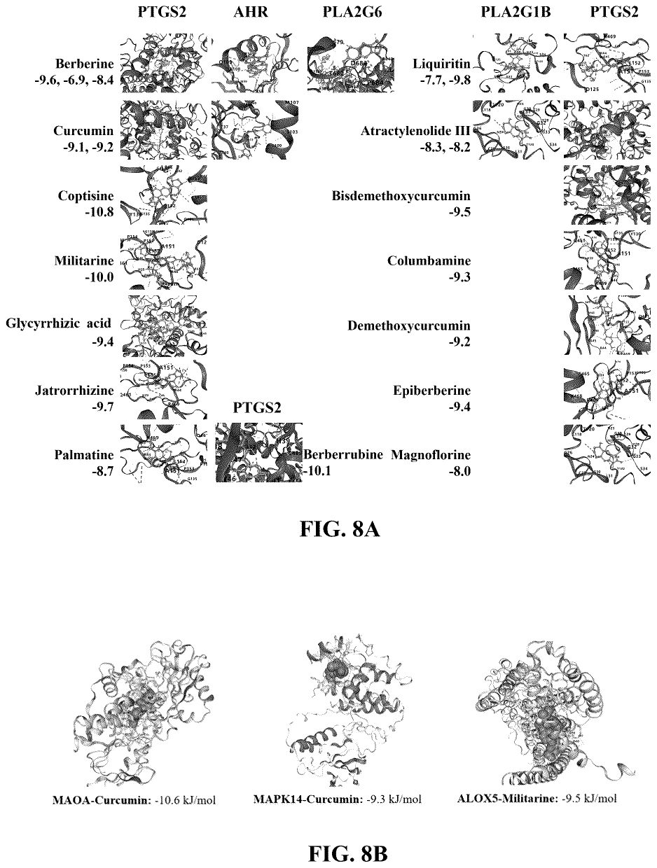

Molecular docking was conducted to assess the interaction between fifteen CMF compounds and key targets (FIGS. 8A and 8B). The docking analysis of AHR showed that berberine had strong affinity −6.9 kJ/mol and the hydrogen-bonding interaction sites were SER24, THR52, and ASN181. The interactions with MAOA showed that curcumin had the strongest affinity −10.6 kJ/mol, and the interaction sites were SER24, TYR69, and ILE180. In addition, curcumin showed very strong affinity with MAPK14 (−9.3 kJ/mol), and the interaction sites were PRO191, TYR200, and TYR258. In the interactions with PTGS2, atractylenolide III exhibited strong affinity and the hydrogen-bonding interaction sites were CYS1036, ASN1039, and GLU1046. For ALOX5, militarine showed strong affinity and the binding sties were ARG37, ASP40, and ASN102. For PLA2G1B, the docking results showed that liquiritin had hydrogen-bonding interactions with ASN24, TYR28, and GLY33 and the energy was −7.7 kJ/mol.

These results showed high affinities among the CMF compounds and the key targets, implying that they performed a key role in treating UC. Moreover, PTGS2 was identified as a hub gene targeted by most CMF compounds.

### 3.3 COX-2 Activity Assay

Inflammation in IBD is associated with elevated prostaglandin levels derived from linoleic acid metabolism. COX-2, encoded by PTGS2, is upregulated during the early stage of inflammation, leading to increased prostaglandin secretion. Network and integrated analysis identified PTGS2 as a key target regulated by most CMF compounds. The inventors therefore validated the effects of CMF on COX-2 by western blotting and enzyme activity assays.

The inhibitory activities of CMF (10 pg/mL), pure chemicals including bisdemethoxycurcumin (5 μM), demethoxycurcumin and curcumin (1.25 μM) and another fourteen compounds (10 μM) from CMF were assessed using a COX-2 inhibitor screening kit (Beyotime Biotechnology, China). Celecoxib (50 nM) was used as the positive control. The IC50 values were calculated with GraphPad Prism 8.0 software according to the formula: inhibitory rate=(RFU_{560, 590} value of control−RFU_{560, 590} value of compounds)/(RFU_{560, 590} value of control−RFU_{560, 590} value of blank)×100%. The tested concentrations of CMF were 2.5, 5, 10, 15, and 20 μg/mL; of berberine, 1.25, 2.5, 5, 10, and 20 μM; and of curcumin, 0.625, 1.25, 2.5, 5, and 10 μM.

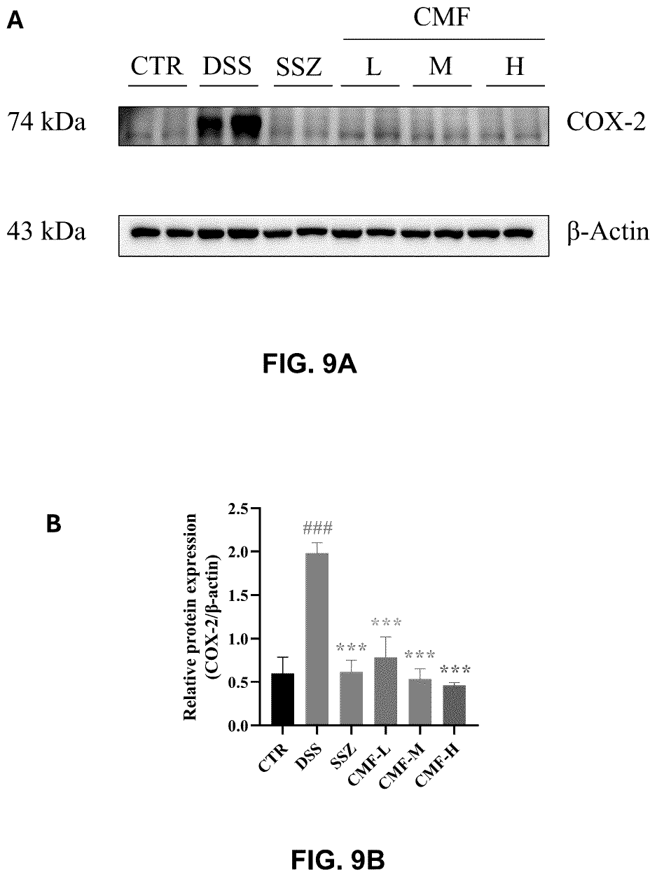

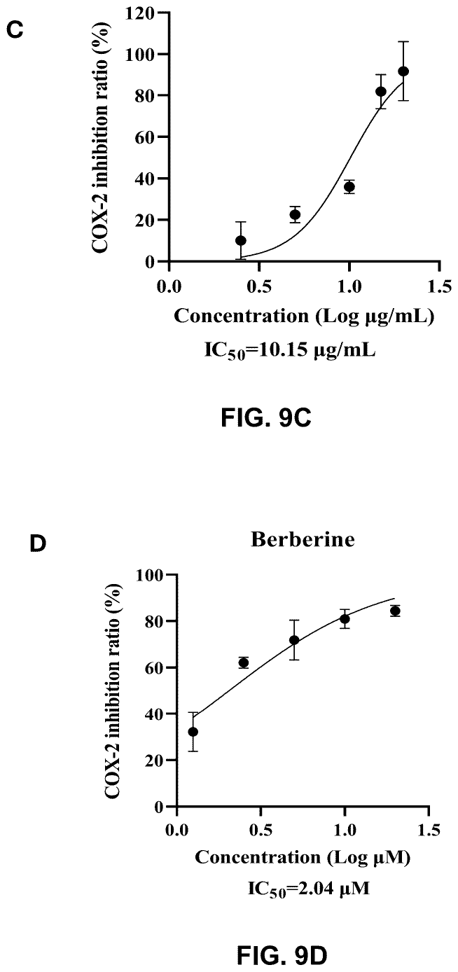

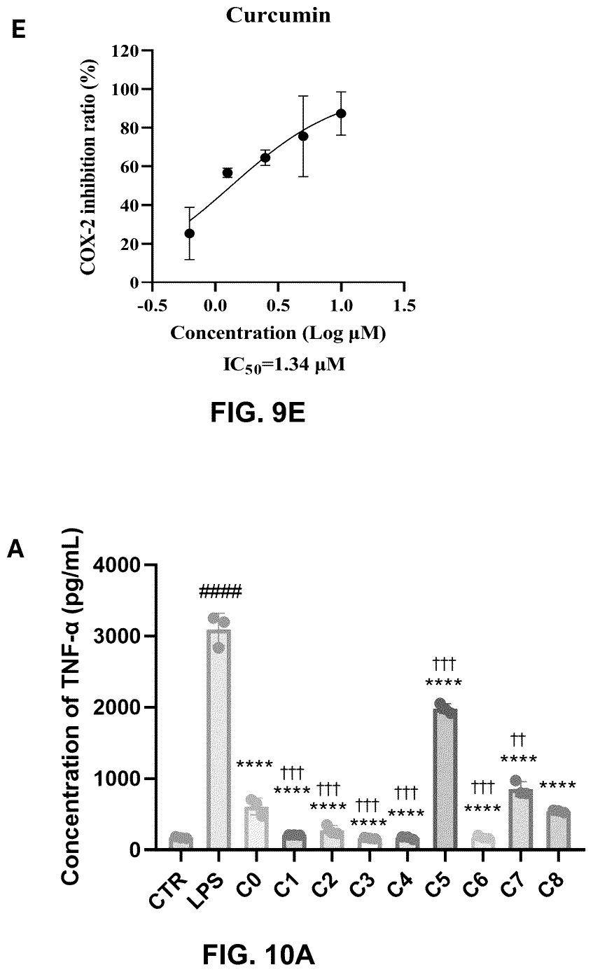

As shown in FIGS. 9A and 9B, the expression of COX-2 was significantly up-regulated in the DSS group, while SSZ and CMF significantly reduced the expression, suggesting they suppress the activation of key target PTGS2. In vitro experiments showed that CMF, along with alkaloids and curcuminoids from HL and JH, exhibited potent COX-2 inhibitory activities, as evidenced by the inhibition rates of CMF (81.8%), epiberberine (70.4%), and bisdemethoxycurcumin (65.5%). Berberine and curcumin were selected as representatives of alkaloids and curcuminoids from HL and JH, respectively. As shown in FIGS. 9C-9E, CMF, berberine, and curcumin inhibited the activity of COX-2 with an IC50 value of 10.15 g/mL, 2.04 M, and 1.34 M, respectively. These results suggest that the anti-colitis effect of CMF may be attributed to inhibiting COX-2.

Immunoblotting and enzyme activity assays provided further evidence that CMF alleviates UC, potentially through its inhibitory effect on cyclooxygenase-2 (COX-2, encoded by PTGS2), with alkaloids and curcuminoids speculated as crucial anti-inflammatory compounds.

The inventors evaluated and studied the identified metabolites, their corresponding metabolic pathways and targets, and their interactions with CMF compounds reveal the anti-colitis mechanism of CMF. Tryptophan metabolites, including host-derived kynine (KA) from the kynurenine pathway and serotonin (5-HT) from the 5-HT pathway, as well as bacterial derivatives in the indole pathway, bind to AHR and exert dual effects on the inflammatory response in inflammatory bowel disease. For example, KA activates GPR35, promotes inflammation, activates AHR, upregulates TSG-6 expression, and enhances the anti-inflammatory effect. Indole derivatives, such as indole pro-ionophilic acid (IPA) as AHR ligands, exhibit significant regulatory performance. The inventors discovered that CMF exerts anti-inflammatory effects through the IL-10 signalling pathway in intestinal epithelial cells. The metabolic process of glycerophospholipids and linoleic acid is a component of lipid metabolism and is essential for maintaining the integrity of the intestinal barrier. Phosphatidylcholine (PC) is the main bioactive phospholipid in mucus. When the phospholipid interacts with PLA2, lysophosphatidylcholine (LPC) and arachidonic acid (AA) are generated. LPC can aggravate the inflammatory response by promoting the polarization reaction of macrophages, while AA can be converted into prostaglandin E2 (PGE2) with pro-inflammatory effects, leading to intestinal irritation, colon muscle relaxation, and diarrhea. In addition, linoleic acid (LA) can also be converted into arachidonic acid (AA), which can serve as a substrate for prostaglandin synthase type 2 (PTGS2). By inhibiting the activity of PTGS2, the production of prostaglandin E2 will be reduced, resulting in an anti-inflammatory effect.

CMF successfully reversed the abnormal metabolic changes caused by KA and IPA in DSS-induced mice. The compounds in CMF also increase phospholipid (PC) levels and reduce elevated lactate phosphate (LPC) and arachidonic acid (AA) levels in mice. Network analysis and molecular docking experiments confirmed the interaction between berberine and curcumin and the AHR (Aromatic Hydrocarbon Receptor), exhibiting exert dual effects on the inflammatory response in inflammatory bowel disease. The inventors also discovered that liquiritin, berberine and atractylenolide III from CMF can bind to the PLA2 enzyme, suggesting that they contribute to alleviating colitis symptoms by regulating the LPC/PC ratio. In addition, militarine and atractylenolide III from CMF exhibited strong binding affinities to ALOX5 and PTGS2, suggesting that they could inhibit the production of inflammatory prostaglandins derived from arachidonic acid (AA).

Based on the results of network analysis and molecular docketing, five active ingredients—berberine, militarine, liquiritin, curcumin and atractylenolide III are confirmed to be the active ingredients from CMF for alleviating UC by affecting the metabolic processes of tryptophan, glycerophospholipids and linoleic acid. Berberine typically and targets AHR; curcumin typically and strongly targets MAOA and MAPK14; atractylenoide III typically targets PTGS2; militarine typically and strongly targets ALOX5; and liquiritin typically and strongly targets PLA2G1B. These active ingredients origins from Coptidis Rhizoma (Huang Lian, HL), Bletillae Rhizoma (Bai Ji, BJ), honey-processed Glycyrrhizae Radix et Rhizoma (Zhi Gan Cao, GC), Curcumae Longae Rhizoma (Jiang Huang, JH), and bran-processed Atractylodis Macrocephalae Rhizoma (Fu Chao Bai Zhu, BZ), respectively. The composition containing the 5 identified active ingredients is herein named SLKK composition.

### Example 4—SLKK Composition Containing the Active Ingredients being Isolated from the Raw Herbs

Taking the formula (1:1:2:2:1 for HL:JH:BJ:BZ:GC respectively) from CMF as a starting point, the inventors evaluated and compared different preparation methods for SLKK.

### 4.1 Extraction of 5 Active Ingredients

To determine how to obtain the five active ingredients from the respective herbs, different extraction methods were evaluated.

SLKK-1 (water extraction): All five herbs, HL, BJ, GC, JH and BZ were boiled with water of 8 times amount for 1 hour twice. The extract was filtered and concentrated under reduced pressure to a thick paste of a relative density of 1.20~1.30 (60° C.). The thick paste was dried under reduced pressure, crushed, and dry paste powder 1 was obtained.

SLKK-2 (water and alcohol precipitation extraction): All five herbs HL, BJ, GC, JH and BZ were boiled with water of 8 times amount for 1 hours twice. The extract was filtered and concentrated under reduced pressure to the relative density of 1.10-1.15 (60° C.) of the paste. After cooling to the room temperature, 95% ethanol was added to make the alcohol content of 70%. The mixture was stirred evenly, left overnight, and the supernatant was concentrated to a thick paste of a relative density of 1.20-1.30 (60° C.). The thick paste was dried under reduced pressure, crushed, and dry paste powder 2 was obtained.

SLKK-3 (water and alcohol extraction): HL (pre-crushed) and JH were reflux extracted by 8 times amount of 70% ethanol for 1 hour twice. The extract was filtered and concentrated under reduced pressure to the relative density of 1.20-1.30 (60° C.) of the concentrate. The thick paste was dried under reduced pressure, crushed, and dry paste powder 1 was obtained. BG, GC, and BZ were boiled with 10 times the amount of water for 1 hour twice. The extract was filtered and concentrated under reduced pressure to the relative density of 1.10-1.15 (60° C.) of the concentrate. The two concentrates were mixed, concentrated under reduced pressure to a thick paste of a relative density of 1.20-1.30 (60° C.). The thick paste was dried under reduced pressure, crushed, and dry paste powder 3 was obtained.

### 4.2 In Vivo Evaluation of Different Dry Paste Powder

The three dry paste powders, SLKK-1, SLKK-2, and SLKK-3, were evaluated in the DSS-induced mouse model as Example 2. 106 C57BL/6J mice were divided into a control group, a DSS group, groups administered with the three dry paste powders, and a positive control group administered with SSZ. The mice of SLKK-administered groups were intragastrically administered with high (11.7 g/kg/day based on raw herbs) and low (23.4 g/kg/day based on raw herbs) doses. Except for the animals in the control group, other groups of mice were administered with 2.5% DSS solution ad libitum to establish an ulcerative colitis mouse model. After the drug intervention, the body weight (Table 5), water intake, disease activity index (DAI) (Table 6), colon length (Table 7), and pathological changes of colon tissue (Tables 8 and 9) were measured or observed.

TABLE 5
Body weight of mice in control group, DSS group, positive control
group, and SLKK-administered groups after administration

| Group | Day 1 | Day 2 | Day 3 | Day 4 | Day 7 | Day 8 |
|---|---|---|---|---|---|---|
| Control group | 18.8 ± 0.8 | 19.0 ± 0.8 | 19.2 ± 0.8 | 19.7 ± 1.1 | 20.3 ± 1.3 | 19.1 ± 0.8 |
| DSS group | 18.6 ± 0.7 | 18.7 ± 0.7 | 18.9 ± 0.7 | 18.9 ± 0.6 | 18.7 ± 0.9^{#} | 18.0 ± 0.9^{#} |
| Positive control | 18.4 ± 0.6 | 18.5 ± 0.6 | 18.8 ± 0.5 | 18.7 ± 0.5 | 18.6 ± 0.6 | 18.0 ± 0.7 |
| group | | | | | | |
| SLKK-1-High | 18.5 ± 0.6 | 18.6 ± 0.6 | 18.8 ± 0.8 | 18.8 ± 0.8 | 18.9 ± 0.9 | 17.8 ± 0.9 |
| SLKK-1-Low | 19.0 ± 1.0 | 19.0 ± 0.9 | 19.0 ± 1.2 | 19.2 ± 1.4 | 19.5 ± 1.4 | 18.4 ± 1.6 |
| SLKK-2-High | 18.7 ± 0.6 | 18.9 ± 0.6 | 19.0 ± 0.7 | 18.9 ± 0.8 | 18.9 ± 0.9 | 18.3 ± 1.0 |
| SLKK-2-Low | 18.8 ± 0.8 | 19.0 ± 0.9 | 19.1 ± 1.0 | 19.2 ± 1.0 | 19.3 ± 1.3 | 18.4 ± 1.6 |
| SLKK-3-High | 18.5 ± 0.8 | 18.6 ± 0.8 | 19.0 ± 0.9 | 19.0 ± 1.0 | 19.0 ± 1.1 | 18.1 ± 1.0 |
| SLKK-3-Low | 18.8 ± 0.5 | 19.1 ± 0.7 | 19.4 ± 0.6 | 19.6 ± 0.6* | 19.7 ± 0.7* | 19.0 ± 0.7* |

^{#}Comparison between CTR group and DSS group, P < 0.05;
*Comparison between positive control/SLKK and DSS groups, P < 0.05.

Compared with the control group, mice in the DSS group were observed to have reduced body weight on Days 7 and 8 after administered with DSS solution (P<0.05). Compared with the DSS group, mice in SLKK-3 groups demonstrated markedly increased body weights on Days 5, 7, and 8 of administration (P<0.05).

TABLE 6
DAI of mice in control group, DSS group, positive control group, and
SLKK-administered groups after administration

| Group | Day 2 | Day 5 | Day 8 |
|---|---|---|---|
| Control group | 0.03 ± 0.18 | 0.00 ± 0.00 | 0.10 ± 0.16 |
| DSS group | 0.50 ± 0.52^{#} | 0.81 ± 2.75^{#} | 1.94 ± 0.75^{#} |
| Positive control | 0.39 ± 0.55 | 0.64 ± 2.57 | 1.25 ± 0.88* |
| group | | | |
| SLKK-1-High | 0.19 ± 0.33 | 0.72 ± 2.04 | 1.69 ± 1.14 |
| SLKK-1-Low | 0.28 ± 0.51 | 0.61 ± 1.47 | 1.73 ± 0.99 |
| SLKK-2-High | 0.19 ± 0.33 | 0.56 ± 2.02 | 1.44 ± 0.62 |
| SLKK-2-Low | 0.31 ± 0.36 | 0.64 ± 2.43 | 1.58 ± 0.91 |
| SLKK-3-High | 0.31 ± 0.36 | 0.36 ± 1.62 | 1.06 ± 0.90* |
| SLKK-3-Low | 0.33 ± 0.38 | 0.58 ± 1.71 | 1.19 ± 0.92* |

^{#}Comparison between CTR group and DSS group, P < 0.05;
*Comparison between positive control/SLKK and DSS groups, P < 0.05.

After Day 5, the stool characteristics from the DSS group altered and the DAI elevated. On day 7, the mice in the DSS group had diarrhea and bloody stools. Compared with the control group, the DSS group showed significantly increased DAI (P<0.05). Both high- and low-dose groups of SLKK-3 and the positive control group significantly reduced DAI in mice (P<0.05).

TABLE 7
Colon length of mice in control group, DSS group, positive control
group, and SLKK-administered groups

| Group | Colon length (cm) |
|---|---|
| Control group | 7.38 ± 0.42 |
| DSS group | 6.17 ± 0.51^{#} |
| Positive control group | 6.73 ± 0.35* |
| SLKK-1-High | 6.47 ± 0.48 |
| SLKK-1-Low | 6.02 ± 0.65 |
| SLKK-2-High | 6.46 ± 0.67 |
| SLKK-2-Low | 6.52 ± 0.63 |
| SLKK-3-High | 7.24 ± 0.47* |
| SLKK-3-Low | 6.41 ± 0.63 |

^{#}Comparison between CTR group and DSS group, P < 0.05;
*Comparison between positive control/SLKK and DSS groups, P < 0.05.

Compared with the control group, the colon length of mice in the DSS group was significantly shortened (P<0.05). SLKK-3-administered groups showed significantly prolonged colon of mice compared with the DSS group. Remarkably, the high dose of SLKK-3 group exhibited even better efficacy than the positive control group.

TABLE 8
Pathological Grading Results for mouse colitis - Degree of epithelial
tissue damage in control group, DSS group, positive control group,
and SLKK-administered groups

| | Dosage | | | | | |
|---|---|---|---|---|---|---|
| | (g/kg based on | Grading | | | | |
| Group | raw herbs) | 0 | 1 | 2 | 3 | 4 |
| Control group | / | 12 | 0 | 0 | 0 | 0 |
| DSS group^{##} | / | 2 | 1 | 0 | 6 | 3 |
| Positive control group | 0.5 | 5 | 1 | 0 | 5 | 1 |
| SLKK-1-High | 23.4 | 2 | 0 | 0 | 6 | 4 |
| SLKK-1-Low | 11.7 | 4 | 0 | 0 | 7 | 0 |
| SLKK-2-High | 23.4 | 6 | 0 | 0 | 4 | 2 |
| SLKK-2-Low | 11.7 | 4 | 4 | 0 | 2 | 2 |
| SLKK-3-High** | 23.4 | 10 | 0 | 0 | 1 | 1 |
| SLKK-3-Low* | 11.7 | 7 | 1 | 0 | 3 | 1 |

^{##}Comparison between CTR group and DSS group, P < 0.01;
*Comparison between positive control/SLKK and DSS groups, P < 0.05;
**Comparison between positive control/SLKK and DSS groups, P < 0.01.

TABLE 9
Pathological Grading Results for mouse colitis - Inflammatory
infiltration in control group, DSS group, positive control group, and
SLKK-administered groups

| | Dosage | | | | | |
|---|---|---|---|---|---|---|
| | (g/kg based | Grading | | | | |
| Group | on raw herbs) | 0 | 1 | 2 | 3 | 4 |
| Control group | / | 12 | 0 | 0 | 0 | 0 |
| DSS group^{##} | / | 2 | 1 | 0 | 6 | 3 |
| Positive control group | 0.5 | 5 | 1 | 0 | 5 | 1 |
| SLKK-1-High | 23.4 | 2 | 7 | 1 | 0 | 1 |
| SLKK-1-Low | 11.7 | 4 | 6 | 2 | 0 | 0 |
| SLKK-2-High | 23.4 | 6 | 4 | 1 | 0 | 1 |
| SLKK-2-Low | 11.7 | 4 | 6 | 1 | 0 | 1 |
| SLKK-3-High** | 23.4 | 10 | 2 | 0 | 0 | 0 |
| SLKK-3-Low* | 11.7 | 7 | 4 | 1 | 0 | 0 |

^{##}Comparison between CTR group and DSS group, P < 0.01;
*Comparison between positive control/SLKK and DSS groups, P < 0.05;
**Comparison between positive control/SLKK and DSS groups, P < 0.01.

In the control group, the colonic mucosa was intact and smooth, with clearly defined glandular layers and no inflammatory cell infiltration. In the DSS group, the colonic mucosal goblet cells were lost, and the crypt structures were disrupted, associated with inflammatory cell infiltration. In the SLKK-administered groups, the colonic mucosal damage was relatively alleviated, the crypts were more regularly arranged, the epithelial structures were relatively intact, and the inflammatory cell infiltration was reduced. Compared with the control group, the grading of colonic tissue lesions in the DSS group was significantly elevated (P<0.01). Compared with the DSS group, the grading of colonic tissue lesions in both the high-dose and low-dose groups of SLKK-3 was significantly reduced (P<0.01 and P<0.05, respectively). These results indicate that SLKK-3 can improve DSS-induced colonic tissue damage.

SLKK-3, which is SLKK prepared by the partial water extraction+partial alcohol extraction, showed ameliorative effect on ulcerative colitis in C57BL/6J mice with the therapeutically effective amount of 11.7-23.4 g/kg/day (based on raw herbs).

### 4.3 Toxicity Study of Single Administration

ICR mice were administered with the three SLKK dry paste powder, SLKK-1, SLKK2, and SLKK-3 by intragastrical gavage in a single administration. There were 4 groups (10 mice in each group, 5 male and 5 female mice): the control group and the groups of three SLKK dry paste powder at 131.5 g/kg/day (based on raw herbs). The mice were observed for 14 consecutive days continuously after administration. The day of the first administration was defined as day 1 (D1). Body weight and food intake of the mice were measured every day, and the mice were sacrificed on day 14.

No mice were dead after administration of three SLKK dry paste powders during the study. The mice administered with the SLKK-3 were observed to show fecal discoloration (drug color) from day 2. The mice administered with SLKK-1 were observed to show fecal discoloration (drug color), watery stools, soft stools, and perianal soiling from day 2. The mice administered with SLKK-2 were observed to show fecal discoloration (drug color), soft stools, and perianal soiling from day 2. All the above symptoms were recovered on day 3 of the study. In addition to the above symptoms, there were no significant alteration in body weight and food intake. No abnormality was observed during the necropsy of the mice at the end of the study.

The result demonstrated that the SLKK compositions prepared in any of formulations of SLKK-1, SLKK-2, and SLKK-3 were safe in vivo, which the maximum tolerable dose (MTD) by intragastrical gavage in a single administration could reach 131.5 g/kg/day (base on raw herbs).

Based on the in vivo evaluation and toxicity study, SLKK-3, which is prepared by partial aqueous extraction+partial water and alcohol extraction, was selected for further optimization.

### 4.4 Berberine and Militarine are Quality Markers for the Composition SLKK

The herbs processed by water extraction are BZ, BJ and GC. The chemical components of BZ are mainly lipophilic components, and the content of the active ingredient, atractylenolide III, is relatively low in BZ. The active ingredient of BJ is militarine, which is also the quality marker for BJ. Therefore, militarine in BJ was selected as the quality marker for the water extraction process to evaluate and optimize the parameters in water extraction as shown in Table 10.

TABLE 10
Content measurement of militarine in batches of SLKK dry paste powders

| SLKK dry | Content of | | | Average | | Average |
|---|---|---|---|---|---|---|
| paste powder | militarine in | Militarine | Transfer | transfer | Extraction | Extraction |
| bathes no. | BJ (%) | (mg/g) | rate (%) | rate (%) | yield (%) | yield (%) |
| JF-221018- | 2.8 | 3.543 | 38.0 | 35.0 | 33.3 | 31.6 |
| 01 | | | | | | |
| JF-221020- | 2.8 | 3.530 | 37.4 | | 33.0 | |
| 02 | | | | | | |
| JF-221020- | 2.8 | 3.528 | 37.7 | | 33.3 | |
| 03 | | | | | | |
| C- | 3.0 | 3.981 | 40.9 | | 34.2 | |
| ZLKYXJCY- | | | | | | |
| 221101 | | | | | | |
| JF-221123- | 2.8 | 3.575 | 38.5 | | 33.5 | |
| 01 | | | | | | |
| JF-221124- | 2.8 | 3.715 | 40.1 | | 33.5 | |
| 02 | | | | | | |
| JF-221125- | 2.8 | 3.451 | 37.1 | | 33.4 | |
| 03 | | | | | | |
| JF-230201 | 2.6 | 2.487 | 22.6 | | 26.3 | |
| JF-230202 | 2.7 | 3.374 | 31.7 | | 28.2 | |
| JF-230203 | 2.6 | 2.772 | 26.1 | | 27.2 | |

The transfer rates of different batches of SLKK dry paste powder varied, but not too much. According to the average transfer rate of 35.0% and average extraction yield of 31.6%, and 1.5% militarine as the quality standard for BJ according to 2020 Chinese pharmacopoeia, the content of militarine is at least 1.48 mg/g in the composition SLKK in the form of dry paste powder.

The herbs processed by water and alcohol extraction are HL and JH. Berberine is one of the main components in HL. It is also the quality marker of HL herbs, which is at least 5.0% for authentication of HL herbs according to the 2020 edition of Chinese Pharmacopoeia. Curcumin is one of the main components in JH. It is also the quality marker of JH herbs, which is at least 0.9% for authentication of JH herbs according to the 2020 edition of Chinese Pharmacopoeia. The content standard of berberine is relatively high, therefore, berberine in HL was selected as the quality marker for the water and alcohol extraction process to evaluate and optimize the parameters in water and alcohol extraction as shown in Table 11.

TABLE 11
Content measurement of berberine hydrochloride in batches of SLKK dry paste powders

| | Content | | | | | |
|---|---|---|---|---|---|---|
| | of | | | | | |
| SLKK dry | berberine | Berberine | | Average | | Average |
| paste powder | hydrochloride | hydrochloride | Transfer | transfer | Extraction | Extraction |
| bathes No. | in HL (%) | (mg/g) | rate (%) | rate (%) | yield (%) | yield (%) |
| JF-221018-01 | 6.8 | 7.578 | 66.9 | | 33.3 | |
| JF-221020-02 | 6.8 | 7.766 | 67.8 | | 33.0 | |
| JF-221020-03 | 6.8 | 7.689 | 67.7 | | 33.3 | |
| C- | 6.5 | 6.577 | 62.3 | | 34.2 | |
| ZLKYXJCY- | | | | | | |
| 221101 | | | | | | |
| JF-221123-01 | 6.8 | 6.722 | 59.7 | 58.9 | 33.5 | 31.6 |
| JF-221124-02 | 6.8 | 6.727 | 59.7 | | 33.5 | |
| JF-221125-03 | 6.8 | 6.864 | 60.7 | | 33.4 | |
| JF-230201 | 7.4 | 6.803 | 43.5 | | 26.3 | |
| JF-230202 | 7.4 | 7.388 | 50.7 | | 28.2 | |
| JF-230203 | 7.5 | 7.716 | 50.4 | | 27.2 | |

The transfer rates of different batches of SLKK dry paste powder varied, but not too much. According to the average transfer rate of 58.9% and average extraction yield of 31.6%, and 5.0% berberine as the quality standard for HL, the content of berberine is at least 4.14 mg/g in the composition SLKK in the form of dry paste powder.

### 4.5 Optimization of the Preparation of the Composition

### 4.5.1 Water Extraction

The water extraction process was based on the amount of water, the number of extractions and the extraction time. A L₉(3⁴) orthogonal test was carried out and the total extraction amount of militarine in the extract was measured as shown in Tables 12 and 13.

TABLE 12
The orthogonal test of parameters in water extraction

| | Parameters | | | | |
|---|---|---|---|---|---|
| | A: water | B: | C: duration | | Extracted |
| | amount | extraction | for each | | militarine |
| No. | (times) | times | extraction | D: blank | (mg) |
| 1 | 1 | 1 | 1 | 1 | 184.1 |
| 2 | 1 | 2 | 2 | 2 | 284.2 |
| 3 | 1 | 3 | 3 | 3 | 310.6 |
| 4 | 2 | 1 | 2 | 3 | 221.4 |
| 5 | 2 | 2 | 3 | 1 | 294.7 |
| 6 | 2 | 3 | 1 | 2 | 336.3 |
| 7 | 3 | 1 | 3 | 2 | 240.5 |
| 8 | 3 | 2 | 1 | 3 | 292.0 |
| 9 | 3 | 3 | 2 | 1 | 346.3 |
| I | 778.9 | 646.0 | 812.4 | 825.1 | G = Σyi = |
| II | 852.4 | 870.9 | 851.9 | 861.0 | 2510.1 |
| III | 878.8 | 993.2 | 845.8 | 824.0 | |
| I² | 606685.2 | 417316.0 | 659993.8 | 680790.0 | GT = G2/9 = |
| II² | 726585.8 | 758466.8 | 725733.6 | 741321.0 | 700066.9 |
| III² | 772289.4 | 986446.2 | 715377.6 | 678976.0 | |
| R | 33.30 | 115.73 | 13.17 | 12.33 | / |
| Q | 701853.5 | 720743.0 | 700368.3 | 700362.3 | |
| S | 1786.6 | 20676.1 | 301.5 | 295.5 | |

TABLE 13
Variance of parameters A, B, and C according to Table 12

| | Sum of squared | Degree of | Mean | | |
|---|---|---|---|---|---|
| | deviations | freedom | square | | |
| Parameter | (S) | (V) | (MS) | F value | Significance |
| A | 1786.58 | 2 | 893.29 | 6.05 | F < 19 |
| B | 20676.13 | 2 | 10338.06 | 69.98 | F > 19 * |
| C | 301.45 | 2 | 150.72 | 1.02 | F < 19 |
| D | 295.45 | 2 | 147.72 | | |

F 0.05(2,2) = 19.00;
F 0.01(2,2) = 99.00.

According to the orthogonal experimental results, the weighing of parameters that affects the total extraction amount of militarine is B>A>C. The extraction times has the greatest impact on the extracted amount of militarine. The analysis of variance shows that the extraction times has a significant difference in the extracted amount of militarine. Accordingly, the preferred water extraction process is A₃B₃C₂, which is: 10 times amount of water added, extraction for 3 times, and the extraction duration of 1.0 hour each time. However, there is no significant difference between 10 times and 8 times amount of water. Thus, 8 times amount of water, extraction for 3 times and the extraction duration of 1.0 hour each time is determined as the optimized water extraction process.

### 4.5.2 Water and Alcohol Extraction

The water and alcohol extraction process was first evaluated by single factor test. The total amount of extracted berberine hydrochloride is the marker. The optimal concentration of ethanol was 7000 ethanol. A L₉(3⁴) orthogonal test was carried out for amount of solvent, extraction times and extraction duration as shown in Tables 14 and 15.

TABLE 14
the orthogonal test of parameters in water and alcohol extraction

| | Parameters | | | | Extracted |
|---|---|---|---|---|---|
| | A: Alcohol | | | | berberine |
| | amount | B: extraction | C: duration for | | hydrochloride |
| No. | (times) | times | each extraction | D: blank | (mg) |
| 1 | 1 | 1 | 1 | 1 | 280.7 |
| 2 | 1 | 2 | 2 | 2 | 423.6 |
| 3 | 1 | 3 | 3 | 3 | 526.2 |
| 4 | 2 | 1 | 2 | 3 | 326.6 |
| 5 | 2 | 2 | 3 | 1 | 505.9 |
| 6 | 2 | 3 | 1 | 2 | 469.2 |
| 7 | 3 | 1 | 3 | 2 | 388.5 |
| 8 | 3 | 2 | 1 | 3 | 412.2 |
| 9 | 3 | 3 | 2 | 1 | 542.0 |
| I | 1230.6 | 995.8 | 1162.1 | 1328.6 | G = Σyi = |
| II | 1301.7 | 1341.7 | 1292.2 | 1281.3 | 3875.0 |
| III | 1342.7 | 1537.5 | 1420.7 | 1265.1 | |
| I² | 1514376.4 | 991617.6 | 1350476.4 | 1765178.0 | GT = G2/9 = |
| II² | 1694422.9 | 1800158.9 | 1669780.8 | 1641729.7 | 1668402.8 |
| III² | 1802843.3 | 2363906.3 | 2018388.5 | 1600478.0 | |
| R | 37.37 | 180.57 | 86.20 | 21.17 | / |
| Q | 1670547.5 | 1718560.9 | 1679548.6 | 1669128.6 | hydrochloride |
| S | 2144.7 | 50158.2 | 11145.8 | 725.8 | (mg) |

TABLE 15
Variance of parameters A, B, and C according to Table 14

| | Sum of squared | Degree of | Mean | | |
|---|---|---|---|---|---|
| | deviations | freedom | square | | |
| Parameter | (S) | (V) | (MS) | F value | Significance |
| A | 2144.74 | 2 | 1072.37 | 2.96 | F < 19 |
| B | 50158.15 | 2 | 25079.07 | 69.11 | F > 19 * |
| C | 11145.80 | 2 | 5572.90 | 15.36 | F < 19 |
| D | 725.78 | 2 | 362.89 | | |

F 0.05(2,2) = 19.00;
F 0.01(2,2) = 99.00

According to the orthogonal experimental results, the weighing of parameters that affects the total extraction amount of berberine hydrochloride is B>A>C. The extraction times has the greatest impact on the extracted amount of berberine hydrochloride. The analysis of variance shows that the extraction times has a significant difference in the extracted amount of berberine hydrochloride. Accordingly, the preferred water extraction process is A₃B₃C₃, which is: 10 times amount of alcohol added, extraction for 3 times, and the extraction duration of 1.5 hour each time. However, there is no significant difference between 10 times and 8 times amount of alcohol, or between 1.5-hour and 1.0-hour extraction duration. Thus, 8 times amount of alcohol, extraction for 3 times and the extraction duration of 1.0 hour each time is determined as the optimized water and alcohol extraction process.

### 4.5.3 Temperature for Concentration

Concentration is an important step for preparing the dry paste powder of the extractions of raw herbs. The temperature during the concentration step is a parameter that may affect the extraction efficiency. Table 16 shows the extracted results at different temperatures in concentration under reduced pressure.

TABLE 16
The extracted amount of militarine and berberine hydrochloride at
different temperatures during concentration step

| | | Berberine hydrochloride |
|---|---|---|
| Temperature ° C. | Militarine (mg) | (mg) |
| Before concentration | 349.8 | 520.7 |
| 60 | 339.8 | 467.7 |
| 70 | 345.9 | 462.3 |
| 80 | 344.0 | 463.5 |

There is no significant difference in extracted amount of militarine and berberine hydrochloride between the extraction before concentration and the concentrates under 60-80° C. The loss of militarine and berberine hydrochloride can be omitted. Thus, the preferred concentrate temperature is not higher than 80° C., or 60-80° C.

The step of removing the solvent from the extraction solvent can be accomplished using well known methods known in the art, such as by evaporation, distillation e.g., under reduced pressure and/or heat, freeze drying, spray drying, fluid bed drying, direct oven heat drying, etc. Regarding drying process, drying under reduced pressure or vacuum belt are preferred.

### 4.6 Validation of the Preparation of SLKK

Based on Example 5.5, three batches of SLKK compositions were prepared. Table 17 shows the results of validation of the optimized preparation of SLKK in three batches.

TABLE 17
Information of three batches of SLKK prepared by the optimized
preparation method

| | JF-221018- | JF-221020- | JF-221020- |
|---|---|---|---|
| Batch no. | 01 | 02 | 03 |
| Raw herbs (kg) | 2.88 | 2.88 | 2.88 |
| water amount (L) | 49.80 | 49.50 | 49.93 |
| alcohol amount (L) | 10.19 | 10.12 | 10.15 |
| Concentrate (kg) | 2.75 | 2.76 | 2.78 |
| Dry paste powder (kg) | 960 | 950 | 958 |
| Extraction yield (paste powder, %) | 33.3 | 33.0 | 33.3 |
| Water content in dry paste powder (%) | 3.0 | 2.9 | 2.9 |
| Berberine hydrochloride (mg/g) | 7.578 | 7.766 | 7.689 |
| Coptisine hydrochloride, palmatine | 4.175 | 4.247 | 4.211 |
| hydrochloride, and epiberberine | | | |
| hydrochloride (mg/g) | | | |
| Militarine (mg/g) | 3.543 | 3.530 | 3.528 |

The three batches of dry paste powder are basically the same, and in good alignment, indicating that the optimized preparation method is stable and feasible.

These results demonstrate that SLKK composition containing the active ingredients being isolated from the raw herbs exhibited significant efficacy in alleviating ulcerative colitis and showed no obvious toxicity. Berberine and militarine are quality markers to confirm the efficacy of SLKK composition.

### Example 5—P-SLKK Composition of 5 Active Ingredients

Based on the results on SLKK composition containing the active ingredients being isolated from the corresponding raw herbs of Example 4, the inventors prepared a composition of berberine, militarine, liquiritin, curcumin and atractylenolide III (P-SLKK) and evaluated the efficacy of P-SLKK.

### 5.1 Component Ratio of Five Active Compounds in P-SLKK

Four batches of SLKK compositions (JF-230201, JF-230202, JF-230203 and SLKK-3) were analyzed for the concentrations of five active ingredients by liquid chromatography-mass spectrometry (Table 18). The composition of SLKK-3 includes 10.42 mg/g berberine, 5.18 mg/g militarine, 2.28 mg/g liquiritin, 0.62 mg/g curcumin, and 0.019 mg/g atractylenolide III, which the ratio is approximately 50:20:10:5:0.1.

TABLE 18
Detected concentrations of five active compounds in different batches of SLKK (mg/g).

| Batch no. | Berberine | Militarine | Liquiritin | Curcumin | Atractylenolide III |
|---|---|---|---|---|---|
| JF-230201 | 9.05 | 2.49 | 1.09 | 0.25 | 0.0096 |
| JF-230202 | 10.09 | 3.37 | 1.43 | 0.73 | 0.032 |
| JF-230203 | 10.75 | 2.78 | 0.98 | 0.28 | 0.019 |
| SLKK-3 | 10.42 | 5.18 | 2.28 | 0.62 | 0.019 |
| Range | 9.05-10.75 | 2.49-5.18 | 0.98-2.28 | 0.25-0.73 | 0.0096-0.032 |

Based on the detected concentrations of each active ingredient in SLKK compositions in Table 18, the inventors prepared a P-SLKK composition at an approximate weigh ratio of 50:20:10:5:0.1 as C0. Other eight compositions of P-SLKK (C1-C8) of different ratios were prepared as shown in Table 19.

TABLE 19
Weight ratios of five active ingredients in P-SLKK compositions
C0-C8

| P-SLKK | Berberine | Militarine | Liquiritin | Curcumin | Atractylenolide III |
|---|---|---|---|---|---|
| C0 | 50 | 20 | 10 | 5 | 0.1 |
| C1 | 65 | 15 | 10 | 5 | 0.1 |
| C2 | 50 | 30 | 10 | 5 | 0.1 |
| C3 | 50 | 20 | 15 | 5 | 0.1 |
| C4 | 50 | 20 | 10 | 8 | 0.1 |
| C5 | 50 | 20 | 10 | 5 | 0.5 |
| C6 | 35 | 30 | 10 | 5 | 0.1 |
| C7 | 50 | 10 | 20 | 5 | 0.1 |
| C8 | 40 | 20 | 10 | 5 | 0.01 |
| wt. % | 43.70%-68.35% | 11.75%-37.45% | 10.52%-23.50% | 5.26%-9.08% | 0.01%-0.58% |

### 5.2 In Vitro Evaluation of P-SLKK Compositions of Different Ratios

To reveal the efficacy of P-SLKK of different ratios, the in vitro anti-inflammatory effect of C0-C8 was evaluated. RAW264.7 cells were cultured in Dulbecco's Modified Eagle Medium containing 10% fetal bovine serum and 100 units/mL penicillin-streptomycin at 37° C. in a 5% CO₂ incubator. Cells were plated in a 96-well plate at a density of 10×10⁶ cells/mL and incubated for 24 h. Further, cells were stimulated with 2 g/mL lipopolysaccharide (LPS) in the presence of P-SLKK of C0-C8 for 24 h. A LPS group was cells stimulated with only LPS (2 g/mL) for 24 h. Control group (CTR) was not stimulated with any extra substances. Subsequently, 100 μL of supernatant of each group was transferred to a 96-well plate, and IL-6 and TNF-α production were tested by the LEGENDplex Mouse Inflammation Panel kit (740150, Biolegend). Inhibition rate was calculated as: (mean LPS cytokine concentration−sample cytokine concentration)/(mean LPS cytokine concentration−mean CTR cytokine concentration)×100%. Data is shown as mean±SD (n=3). Two-group comparisons were performed with Student's t-test; multiple groups were compared by one-way ANOVA.

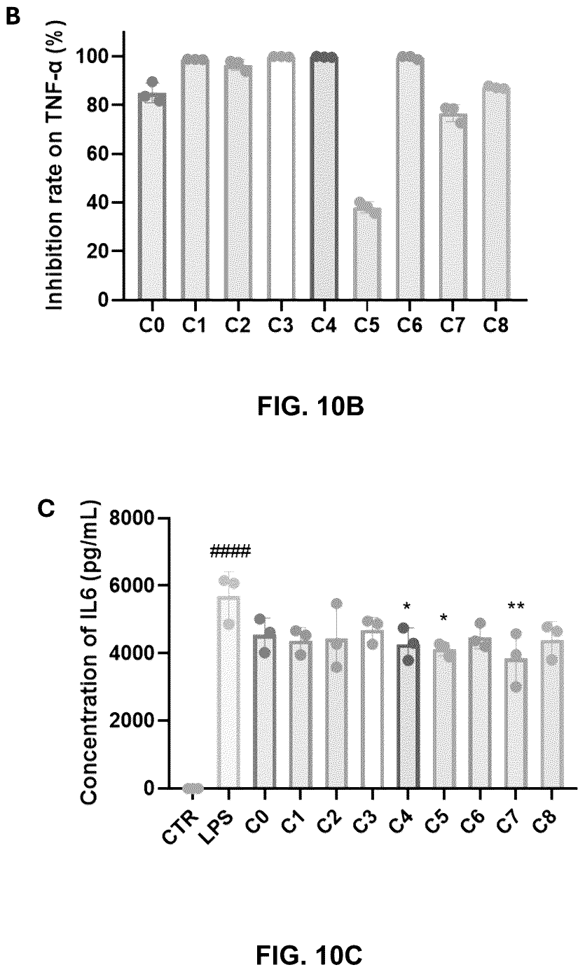

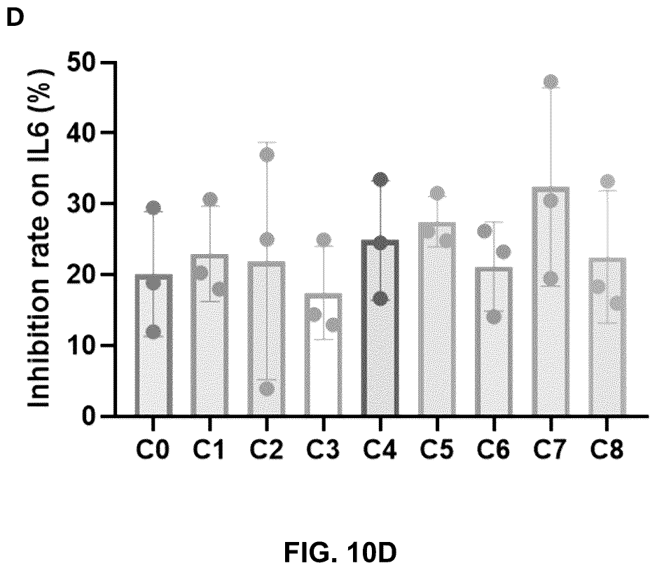

As shown in FIG. 10A, LPS stimulation significantly increased TNF-α production compared to the control group (3093 vs. 170 pg/mL, p<0.001), confirming successful establishment of the inflammatory model. All P-SLKK groups significantly inhibited LPS-induced TNF-α production (FIG. 10B). The results demonstrated that P-SLKK compositions exhibited significant inhibitory efficacy against TNF-α (p<0.0001).

Compared with C0 as the baseline formulation, C1-C4, and C6 exhibited enhanced efficacy (inhibition rates: 96.5-99.9%). In contrast, C5 (atractylenolide III increased to 0.58%) showed markedly reduced efficacy (inhibition rate: 38.1%), suggesting a potential antagonistic effect of high dose atractylenolide III. Moreover, C7 (liquiritin increased to 23.50%) also showed significantly lower efficacy (inhibition rate: 76.7%).

LPS stimulation also significantly increased IL-6 production compared to the control group (FIG. 10C). All P-SLKK groups markedly reduced the high secretion of IL-6 induced by LPS, especially in C4, C5, and C7 groups (FIG. 10D). There was no significant difference in the inhibitory effect against IL-6 among all P-SLKK groups (p=0.757).

TNF-α and IL-6 are key cytokines in UC. These results suggested that P-SLKK exhibited the anti-inflammatory effects against TNF-α and IL-6 in the LPS-induced inflammatory model, which indicates that P-SLKK is the combination of the active ingredients of CMF.

Although the invention has been described in terms of certain embodiments, other embodiments apparent to those of ordinary skill in the art are also within the scope of this invention. Accordingly, the scope of the invention is intended to be defined only by the claims which follow.

What is claimed is:

1. A pharmaceutical composition comprises 40-70 wt. % berberine, 10-40 wt. % militarine, 10-25 wt. % liquiritin, 5-10 wt. % curcumin, and 0.01-1 wt. % atractylenolide III being characterized by molecular docking with an ulcerative colitis (UC)-modulating target selected from the group consisting of MAOA, MAPK14, AHR, PTGS2, PLA2G1B, ALOX5, and a combination thereof.

2. The pharmaceutical composition of claim 1, wherein berberine, militarine, liquiritin, curcumin and atractylenolide III are respectively isolated from Coptidis Rhizoma, Bletillae Rhizoma, honey-processed Glycyrrhizae Radix et Rhizoma, Curcumae Longae Rhizoma, and bran-processed Atractylodis Macrocephalae Rhizoma.

3. The pharmaceutical composition of claim 2, wherein berberine is at least 4.14 mg/g in the pharmaceutical composition.

4. The pharmaceutical composition of claim 2, wherein militarine is at least 1.48 mg/g in the pharmaceutical composition.

5. The pharmaceutical composition of claim 2, wherein berberine is 4.14-10.41 mg/g in the pharmaceutical composition.

6. The pharmaceutical composition of claim 2, wherein militarine is 1.48-5.71 mg/g in the pharmaceutical composition.

7. The pharmaceutical composition of claim 1, wherein berberine targets AHR, curcumin targets MAOA and MAPK14, atractylenoide III targets PTGS2, militarine targets ALOX5 and liquiritin targets PLA2G1B.

8. The pharmaceutical composition of claim 1, comprises 43.70-68.35 wt. % berberine, 11.75-37.45 wt. % militarine, 10.52-23.50 wt. % liquiritin, 5.26-9.08 wt. % curcumin, and 0.01-0.58 wt. % atractylenolide III.

9. The pharmaceutical composition of claim 1, wherein berberine, militarine, liquiritin, curcumin and atractylenolide III are in weight ratio of 50:20:10:5:0.1.

10. The pharmaceutical composition of claim 1, wherein a therapeutically effective amount of the pharmaceutical composition is 5.8-23.4 g/kg.

11. A method of treating or alleviating ulcerative colitis (UC) in a subject in need thereof, comprises administering a therapeutically effective amount of a composition consisting of berberine, militarine, liquiritin, curcumin and atractylenolide III being characterized by molecular docking with the UC-modulating targets consisting of MAOA, MAPK14, AHR, PTGS2, PLA2G1B, and ALOX5 to the subject.

12. The method of claim 11, wherein the composition consists of 40-70 wt. % berberine, 10-40 wt. % militarine, 10-25 wt. % liquiritin, 5-10 wt. % curcumin and 0.01-1 wt. % atractylenolide III.

13. The method of claim 12, wherein the composition consists of 43.70-68.35 wt. % berberine, 11.75-37.45 wt. % militarine, 10.52-23.50 wt. % liquiritin, 5.26-9.08 wt. % curcumin, and 0.01-0.58 wt. % atractylenolide III.

14. The method of claim 11, wherein berberine, militarine, liquiritin, curcumin and atractylenolide III are in weight ratio of 50:20:10:5:0.1.

15. The method of claim 11, wherein the therapeutically effective amount is 5.8-23.4 g/kg in a single dose or multiple doses daily.

16. The method of claim 11, wherein the composition is administered via intragastrical route.

17. The method of claim 11, wherein the subject is a human, a nonhuman primate, a farm animal or a domesticated mammal, or a laboratory animal.

18. A method of preparing the pharmaceutical composition of claim 1 for treating or alleviating ulcerative colitis (UC), comprising:
(a) characterizing which ingredients from a plurality of ingredients as active ingredients of the pharmaceutical composition by molecular docketing with the UC-modulating targets consisting of MAOA, MAPK14, AHR, PTGS2, PLA2G1B, and ALOX5;
(b) selecting berberine, militarine, liquiritin, curcumin and atractylenolide III based on the molecular docketing results obtained in (a); and
(c) providing and mixing 40-70 wt. % berberine, 10-40 wt. % militarine, 10-25 wt. % liquiritin, 5-10 wt. % curcumin and 0.01-1 wt. % atractylenolide III.

19. The method of claim 18, wherein militarine, liquiritin, and atractylenolide III are isolated from Bletillae Rhizoma, honey-processed Glycyrrhizae Radix et Rhizoma and bran-processed Atractylodis Macrocephalae Rhizoma by water extraction respectively; and berberine and curcumin are isolated from Coptidis Rhizoma and Curcumae Longae Rhizoma by alcohol extraction respectively.

20. The method of claim 19, wherein the water extraction is extracting 3 times with 8-10 times amount of water at a duration of 1 hour, and the alcohol extraction is extracting 3 times with 8-10 times amount of 70% ethanol at a duration of 1-1.5 hours.
